(19) United States
(12) Patent Application Publication
(10) Pub. No.: US 2022/0089646 A1
(43) Pub. Date: Mar. 24, 2022

(54) A CYCLIC PEPTIDE

(71) Applicants: THE UNIVERSITY OF QUEENSLAND, St. Lucia, Queensland (AU); PREVECEUTICAL MEDICAL INC, Vancouver (CA)

(72) Inventors: Harendra PAREKH, Woolloongabba, Queensland (AU); Peter CABOT, St Lucia, Queensland (AU); Karnaker TUPALLY, St Lucia, Queensland (AU); Ajit KANDALE, St Lucia, Queensland (AU)

(21) Appl. No.: 17/424,649

(22) PCT Filed: Jan. 24, 2020
(86) PCT No.: PCT/AU2020/050049

§ 371 Date: Jul. 21, 2021

(30) Foreign Application Priority Data
Jan. 24, 2019 (AU) 2019900226
Jan. 31, 2019 (AU) 2019900292

(51) Int. Cl. C07K 7/64; A61P 25/04

(52) U.S. Cl. CPC C07K 7/64; A61K 38/00; A61P 25/04

(57) ABSTRACT

The present invention provides for novel cyclized peptides which may be useful in the treatment and/or prevention of pain in a subject.

### FIELD OF THE INVENTION

The present invention relates to cyclized peptides. More particularly, the invention relates to cyclized peptides and their use in pain management. Most particularly, the invention relates to cyclized dynorphin analogues and their use in pain management.

### BACKGROUND TO THE INVENTION

Any reference to background art herein is not to be construed as an admission that such art constitutes common general knowledge in Australia or elsewhere.

Opioids are a class of drugs that are used clinically as painkillers. As such, opioids are a mainstay of pain management. However, opioids such as morphine have significant side-effects including constipation, sedation, respiratory depression, dependence and tolerance. These side-effects add significant burden to the quality of life experienced by patients, with prevention and management of opioid dependence being particularly challenging.

Opioids mainly act via the opioid receptors (μ, δ, κ and nociceptin). It is postulated that some of the side-effects reside in the agonist activity on some of these opioid receptors. As such, it would be advantageous to provide an opioid that has selective activity on some receptors to ameliorate this issue.

The natural mechanism for analgesia involves endogenous opioids. One such endogenous opioid is dynorphin which arises from prodynorphin. However, dynorphins are metabolised relatively quickly and so it would be advantageous to provide dynorphins which have greater pharmacokinetic (metabolic) stability and thus a longer half-life.

In one aspect, it should be clear that there is a need for the development of new drugs that are effective in pain management. It would also be advantageous if these new drugs could demonstrate reduced side-effects. It would also be advantageous if these new drugs exhibited greater stability. Alternatively, it would be desirable to have a larger selection of drugs for pain management to choose from.

In another aspect, there is a need for the development of peptidic compounds that exhibit improved stability.

### SUMMARY OF THE INVENTION

In a first aspect, although it need not be the only or indeed the broadest form, the invention resides in a compound of formula (I), or a salt or stereoisomer or solvate or prodrug thereof:

X₁-X₂-X₃-X₄-X₅-X₆-X₇-X₈-X₉-X₁₀-X₁₁ Formula (I)

wherein X₁, X₃, X₄, X₅, X₆ and X₇ are each independently an amino acid or derivative thereof; wherein X₂, X₈, X₉, X₁₀ and X₁₁, when present, are each independently an amino acid or derivative thereof; and

wherein a pair of any of X₁, X₂, X₃, X₄, X₅, X₆, X₇, X₈, X₉, X₁₀ and X₁₁ together form a linker

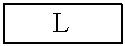

comprising

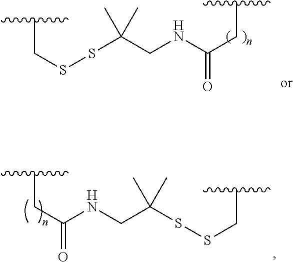

wherein n is 1 or 2.

In one embodiment,

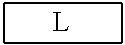

is formed between X₂ or X₃ and any one of X₁, X₄, X₅, X₆, X₇, X₈, X₉, X₁₀ and X₁₁.

In another embodiment,

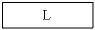

is formed between X₂ and X₅.

In another embodiment,

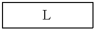

is formed between X₂ or X₃ and X₅.

In another embodiment,

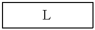

is formed between X₈ and X₁₀.

In another embodiment,

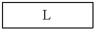

is formed between X₈ and X₉.

In one embodiment, X₁₀ and X₁₁ are not present.

In another embodiment, X₈, X₉, X₁₀ and X₁₁ are not present.

In another embodiment, X₂ is not present.

In an embodiment, n is 1. In another embodiment, n is 2.

In yet another embodiment, the invention resides in a compound of formula (II), or a salt or stereoisomer or solvate or prodrug thereof:

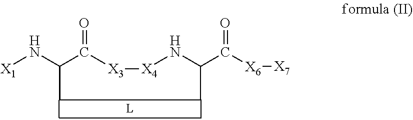

wherein X₁, X₃, X₄, X₆ and X₇ are each independently an amino acid or derivative thereof;
wherein

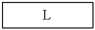

comprises

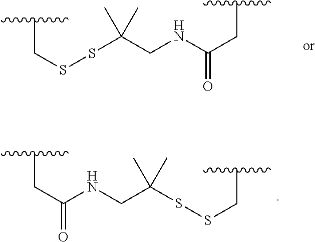 [SMILES: *CSSC(C)(C)CNC(=O)C* ; *CSSC(C)(C)CNC(=O)C*]

In another embodiment, the invention resides in a compound of formula (IX), or a salt or stereoisomer or solvate or prodrug thereof:

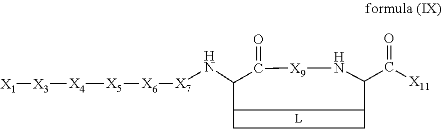

wherein X₁, X₃, X₄, X₅, X₆, X₇, X₉ and X₁₁ are each independently an amino acid or derivative thereof;
wherein

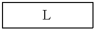

comprises

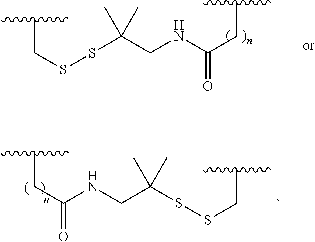

wherein n is 1 or 2.

In one embodiment of the compound of formula (IX),

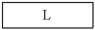

comprises

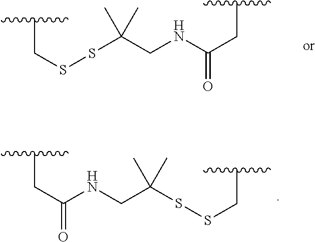 [SMILES: *CSSC(C)(C)CNC(=O)C* ; *CSSC(C)(C)CNC(=O)C*]

In one embodiment of the compounds of formula (I), (II) or (IX), where applicable, X₁, X₂, X₃, X₄, X₅, X₆, X₇, X₈, X₉, X₁₀ and X₁₁ are each independently an L-amino acid or derivative thereof.

In one embodiment of the compounds of formula (I), (II) or (IX), where applicable, X₁, X₂, X₃, X₄, X₅, X₆, X₇, X₈, X₉, X₁₀ and X₁₁ are each independently selected the group consisting of Tyr, Gly, Phe, Leu, Arg, Ile, Pro and Lys.

In one embodiment of the compounds of formula (I), (II) or (IX), where applicable: X₁ is tyrosine or a derivative thereof; X₄ is phenylalanine or a derivative thereof; X₅ is selected from the group consisting of: leucine or a derivative thereof, isoleucine or a derivative thereof, and valine or a derivative thereof; X₆ is arginine or a derivative thereof; and X₇ is arginine or a derivative thereof.

In embodiments of the compounds of formula (I), (II) or (IX), one or more of the following may apply:
where applicable, X₁ may be tyrosine or a derivative thereof, especially L-tyrosine.
where applicable, X₂ may be glycine or a derivative thereof (especially N-alkyl glycine (more especially sarcosine)), or is absent.
where applicable, X₃ may be glycine or a derivative thereof, especially N-alkyl glycine (more especially sarcosine).
where applicable, X₄ may be phenylalanine optionally substituted by one or more of halo (especially chloro or fluoro), or nitro; especially phenylalanine substituted by chloro or nitro. The phenylalanine may be substituted in any suitable position, especially on the phenyl group, more especially at a para position on the phenyl group. The optionally substituted phenylalanine may be optionally substituted L-phenylalanine. In one embodiment, X₄ may be phenylalanine or a derivative thereof.
where applicable, X₅ may be leucine or a derivative thereof; especially leucine. X₅ may be L-leucine or D-leucine; especially L-leucine.
where applicable, X₆ may be arginine or N(α)-alkyl arginine (especially N(α)-methyl arginine); especially arginine. X₆ may be L-arginine, D-arginine, N(α)-alkyl L-arginine (especially N(α)-methyl L-arginine), or N(α)-alkyl D-arginine (especially N(α)-methyl D-arginine); especially L-arginine. In one embodiment, X₆ may be arginine or a derivative thereof.
where applicable, X₇ may be arginine or N(α)-alkyl arginine (especially N(α)-methyl arginine); especially arginine. X₇ may be L-arginine, D-arginine, N(α)-alkyl L-arginine (especially N(α)-methyl L-arginine), or N(α)-alkyl D-arginine (especially N(α)-methyl D-arginine); especially D-arginine or N(α)-alkyl L-arginine (especially N(α)-methyl L-arginine). In one embodiment, X₇ may be arginine or a derivative thereof.
where applicable, X₈ may be isoleucine or a derivative thereof, leucine or a derivative thereof, valine or a derivative thereof, phenylalanine or a derivative thereof, alanine or a derivative thereof, or may be absent. In one embodiment, X₈ may be isoleucine, especially L-isoleucine or D-isoleucine, more especially L-isoleucine.
where applicable, X₈ may be arginine or a derivative thereof or may be absent; especially L-arginine or D-arginine.
where applicable, X₁₀ may be proline or a derivative thereof; especially L-proline.
where applicable, X₁₁ may be lysine or a derivative thereof; especially L-lysine or D-lysine.

In certain embodiments of the compounds of formula (I), (II) or (IX), where applicable, X₁ is tyrosine, and X₆ and X₇ are independently arginine or N-alkyl arginine; especially X₁ is L-tyrosine, and X₆ and X₇ are independently L-arginine, D-arginine, N-methyl L-arginine, or N-methyl D-arginine.

In one embodiment, the compound is selected from the group consisting of:

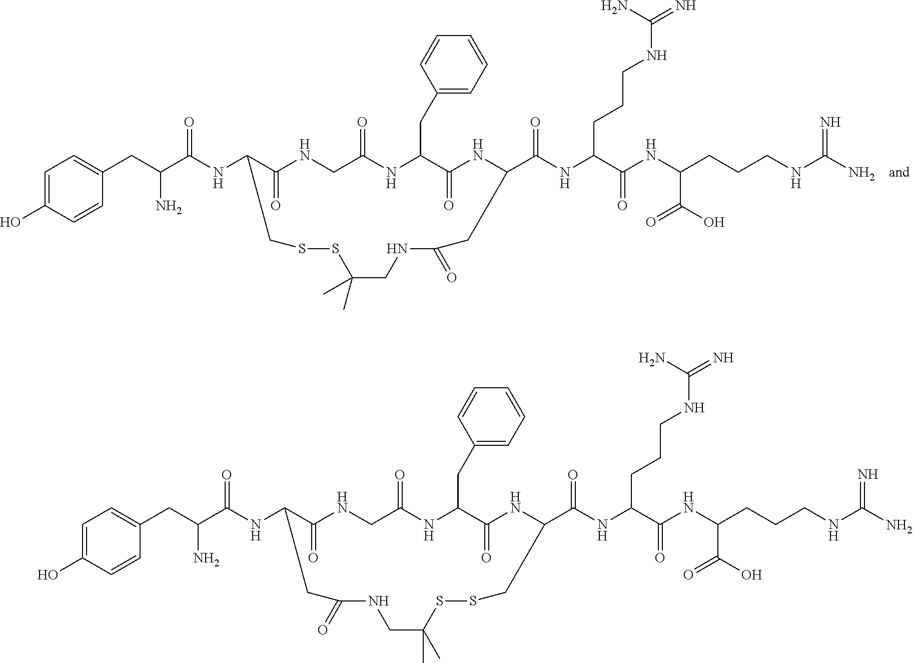 [SMILES: CC1(C)CNC(=O)CC(C(=O)NC(CCCNC(=N)N)C(=O)NC(CCCNC(=N)N)C(=O)O)NC(=O)C(Cc2ccccc2)NC(=O)CNC(=O)C(NC(=O)C(N)Cc2ccc(O)cc2)CSS1 ; CC1(C)CNC(=O)CC(NC(=O)C(N)Cc2ccc(O)cc2)C(=O)NCC(=O)NC(Cc2ccccc2)C(=O)NC(C(=O)NC(CCCNC(=N)N)C(=O)NC(CCCNC(=N)N)C(=O)O)CSS1]

or a salt or stereoisomer or solvate or prodrug thereof.

In an embodiment, the compound is selected from the group consisting of:

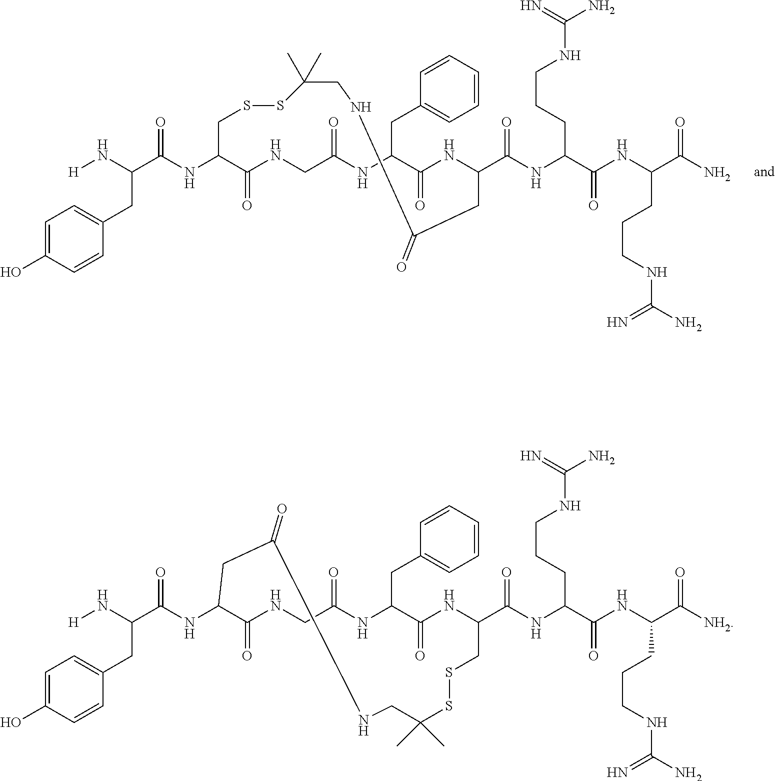 [SMILES: CC1(C)CNC(=O)CC(C(=O)NC(CCCNC(=N)N)C(=O)NC(CCCNC(=N)N)C(N)=O)NC(=O)C(Cc2ccccc2)NC(=O)CNC(=O)C(NC(=O)C(N)Cc2ccc(O)cc2)CSS1 ; CC1(C)CNC(=O)CC(NC(=O)C(N)Cc2ccc(O)cc2)C(=O)NCC(=O)NC(Cc2ccccc2)C(=O)NC(C(=O)NC(CCCNC(=N)N)C(=O)N[C@@H](CCCNC(=N)N)C(N)=O)CSS1]

In another embodiment, the compound is selected from the group consisting of:

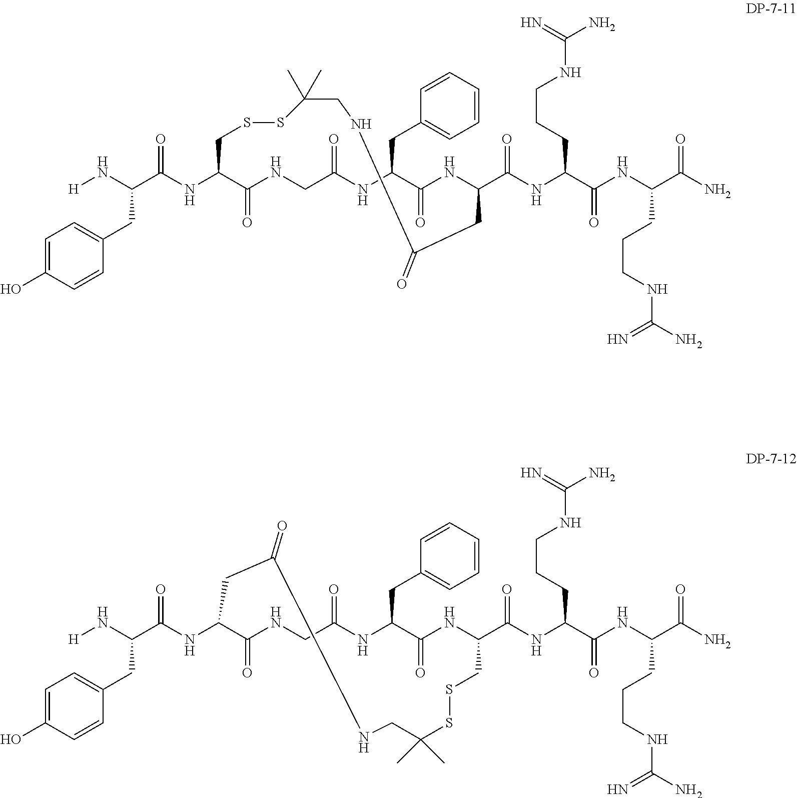 [SMILES: CC1(C)CNC(=O)C[C@H](C(=O)N[C@@H](CCCNC(=N)N)C(=O)N[C@@H](CCCNC(=N)N)C(N)=O)NC(=O)[C@H](Cc2ccccc2)NC(=O)CNC(=O)[C@@H](NC(=O)[C@@H](N)Cc2ccc(O)cc2)CSS1 ; CC1(C)CNC(=O)C[C@@H](NC(=O)[C@@H](N)Cc2ccc(O)cc2)C(=O)NCC(=O)N[C@@H](Cc2ccccc2)C(=O)N[C@H](C(=O)N[C@@H](CCCNC(=N)N)C(=O)N[C@@H](CCCNC(=N)N)C(N)=O)CSS1]

In a further embodiment, the compound is selected from the group consisting of:

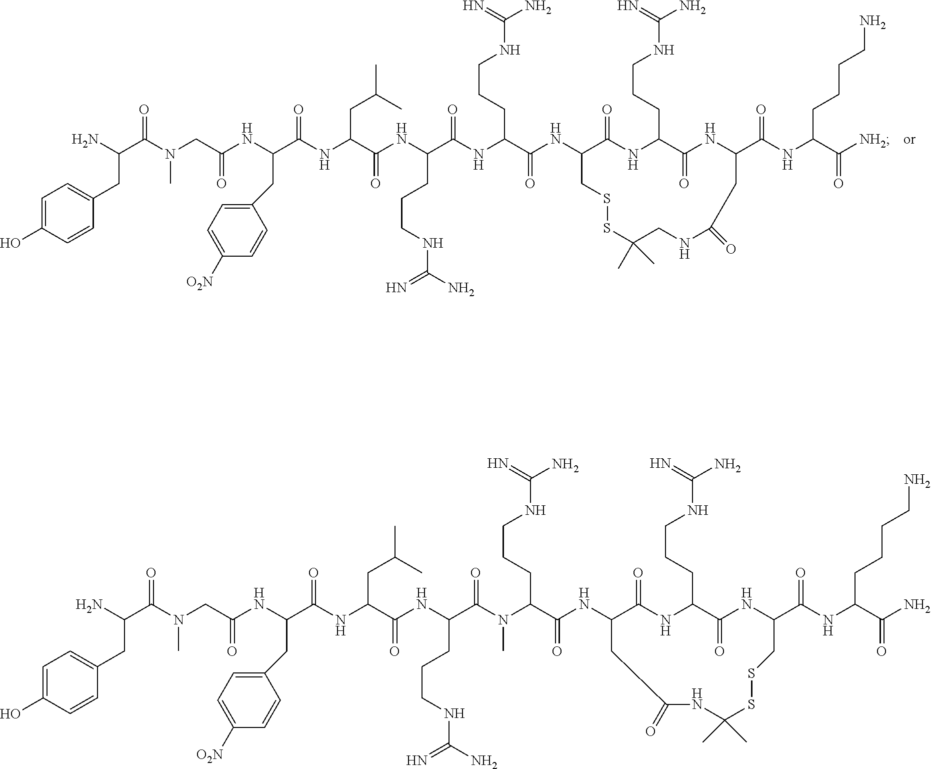 [SMILES: CC(C)CC(NC(=O)C(Cc1ccc([N+](=O)[O-])cc1)NC(=O)CN(C)C(=O)C(N)Cc1ccc(O)cc1)C(=O)NC(CCCNC(=N)N)C(=O)NC(CCCNC(=N)N)C(=O)NC1CSSC(C)(C)CNC(=O)CC(C(=O)NC(CCCCN)C(N)=O)NC(=O)C(CCCNC(=N)N)NC1=O ; CC(C)CC(NC(=O)C(Cc1ccc([N+](=O)[O-])cc1)NC(=O)CN(C)C(=O)C(N)Cc1ccc(O)cc1)C(=O)NC(CCCNC(=N)N)C(=O)N(C)C(CCCNC(=N)N)C(=O)NC1CC(=O)NC(C)(C)SSCC(C(=O)NC(CCCCN)C(N)=O)NC(=O)C(CCCNC(=N)N)NC1=O]

or a salt or stereoisomer or solvate or prodrug thereof.

In another embodiment, the compound is:

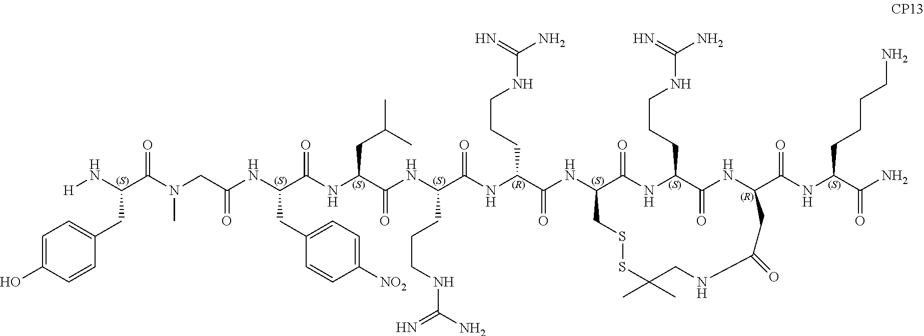

or a salt or solvate thereof.

In another embodiment, the compound is:

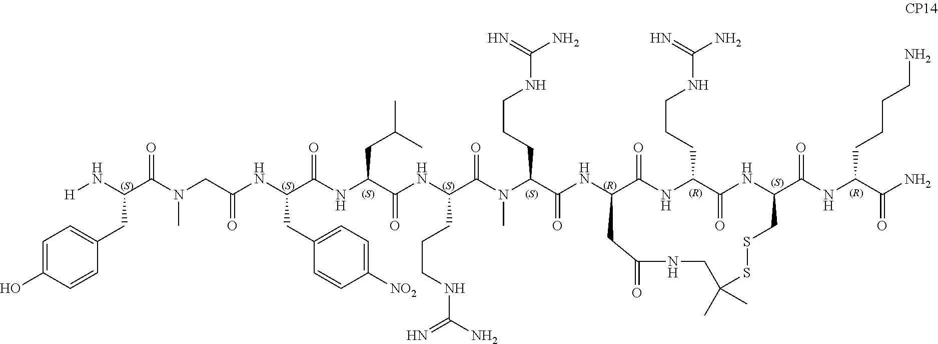

or a salt or solvate thereof.

In a second aspect, the present invention relates to a compound of formula (I), or a salt or stereoisomer or solvate or prodrug thereof:

X₁-X₂-X₃-X₄-X₅-X₆-X₇-X₈-X₉-X₁₀-X₁₁ Formula (I)

wherein X₁, X₂, X₃, X₄, X₅, X₆ and X₇ are each independently an amino acid; wherein X₈, X₉, X₁₀ and X₁₁, when present, are each independently an amino acid; and

wherein a pair of any of X₁, X₂, X₃, X₄, X₅, X₆, X₇, X₈, X₉, X₁₀ and X₁₁ together form a linker

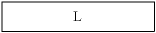

comprising

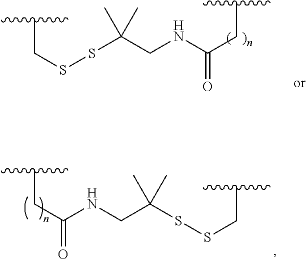

wherein n is 1 or 2.

In a fourth aspect, the invention resides in a pharmaceutical composition comprising a compound of the present invention or a pharmaceutically acceptable salt, stereoisomer, solvate or prodrug thereof, and a pharmaceutically acceptable carrier, diluent and/or excipient.

In a fifth aspect, the invention resides in a method of treating or preventing pain in a subject including the step of administering a therapeutically effective amount of a compound of the present invention, or a pharmaceutically acceptable salt, stereoisomer, solvate or prodrug thereof, or the pharmaceutical composition of the fourth aspect, to the subject to thereby treat or prevent pain.

In a sixth aspect, the invention resides in the use of a compound of the present invention, or a pharmaceutically acceptable salt, stereoisomer, solvate or prodrug thereof, or the pharmaceutical composition of the fourth aspect, in the manufacture of a medicament for the treatment or prevention of pain.

In a seventh aspect, the invention resides in a compound of the present invention, or a pharmaceutically acceptable salt, stereoisomer, solvate or prodrug thereof, or the pharmaceutical composition of the fourth aspect, for use in the treatment or prevention of pain.

In an eighth aspect, the present invention provides a molecule comprising a compound of the present invention. For example in the molecule of the eighth aspect, further amino acids may be appended to the N- or C-terminus of the compound of formula (I).

The various features and embodiments of the present invention referred to in the individual sections above and in the description which follows apply, as appropriate, to other sections, mutatis mutandis. Consequently, features specified in one section may be combined with features specified in other sections as appropriate.

Further features and advantages of the present invention will become apparent from the following detailed description.

### BRIEF DESCRIPTION OF THE DRAWINGS

To assist in understanding the invention and to enable a person skilled in the art to put the invention into practical effect, the invention will be described by way of example only with reference to the accompanying drawings, in which:

FIG. 1 indicates the serum stability of DP-7-00;

FIG. 2 indicates the serum stability of DP-7-11;

FIG. 3 indicates the trypsin stability of DP-7-00;

FIG. 4 indicates the trypsin stability of DP-7-11 FIG. 5 is a graphical representation of a sample Forskolin treatment response in HEK-DOP;

FIG. 6 is a graphical representation of a sample Forskolin treatment response in HEK-KOP;

FIG. 7 is a graphical representation of the cAMP standard curve for HEK-DOP;

FIG. 8 is a graphical representation of the cAMP standard curve for HEK-KOP;

FIG. 9 is a graphical representation of cAMP inhibition of DP-7-11 at KOP and DOP;

FIG. 10 is a graphical representation of cAMP inhibition of DP-7-12 at KOP and DOP;

FIG. 11 shows a comparison of IC50 values between DP-7-11 and DP-7-12 in HEK-DOP;

FIG. 12 shows a comparison of IC50 values between DP-7-11 and DP-7-12 in HEK-KOP;

FIG. 13 is a graphical representation of the effect of Naloxone (100 microM) on cAMP inhibition of DP-7-11 and DP-7-12 in HEK-DOP;

FIG. 14 is a graphical representation of the effect of Naloxone (100 microM) on cAMP inhibition of DP-7-11 and DP-7-12 in HEK-KOP;

FIG. 15 indicates the serum stability of DP-11-00;

FIG. 16 indicates the serum stability of DP-11-06;

FIG. 17 indicates the trypsin stability of DP-11-00;

FIG. 18 indicates the trypsin stability of DP-11-06;

FIG. 19 is a series of graphical representations of dose-response curves of peptide KOR agonists in cAMP inhibition (EC₅₀ reported in Table 10 derived from these curves. Data normalised to U50488H as reference compound (max response of which equals 100%). Data fitted to a four-parameter non-linear regression in Prism software. Number of repeats (each in duplicate) noted in title of each curve. Mean+/−SEM);

FIG. 20 is a series of graphs showing the stability of selected peptides in a trypsin (left-hand graphs) and plasma (right-hand graphs) stability assay (data normalised to concentration of each peptide at t=0, determined by LCMS, within each substrate; data analysed using One Phase Decay nonlinear regression in Prism software and each experiment performed in triplicate; mean+/−SEM);

FIG. 21 shows the results of stability screening of select peptides in cAMP buffer (data normalised to concentration of each peptide at t=0, determined by LCMS, within each substrate; data analysed using One Phase Decay nonlinear regression in Prism software; Each experiment performed in triplicate. Mean+/−SEM); and

FIG. 22 shows a graphical representation of a dose-response curve of peptide DP-11-06 in cAMP inhibition (EC₅₀ reported in Table 10 is derived from this curve. Data normalised to U50488H as reference compound (max response of which equals 100%). Data fitted to a four-parameter non-linear regression in Prism software. Mean+/−SEM).

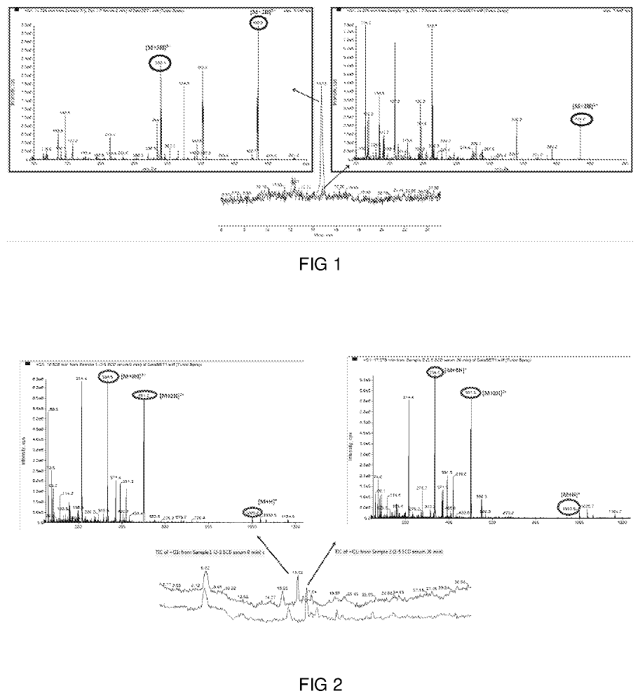

### DETAILED DESCRIPTION OF THE INVENTION

Embodiments of the present invention reside primarily in cyclized peptides. These cyclized peptides may be viewed as dynorphin analogues comprising a cyclic structure.

### Definitions

In this specification, adjectives such as one or more, at least, and the like may be used solely to distinguish one element or action from another element or action without necessarily requiring or implying any actual such relationship or order.

In this specification, the terms ‘comprises’, ‘comprising’, ‘includes’, ‘including’, or similar terms are intended to mean a non-exclusive inclusion, such that a method or groups that comprises a list of steps or elements does not include those steps or elements solely, but may well include other steps or elements not expressly listed.

As used herein, the term ‘about’ means the amount is nominally the number following the term ‘about’ but the actual amount may vary from this precise number to an unimportant degree.

The term ‘amino acid’ refers to naturally-occurring α-amino acids and their stereoisomers. The term ‘stereoisomers’ of amino acids refers to mirror image isomers of the amino acids, such as L-amino acids or D-amino acids. Non-limiting examples of amino acids include alanine (Ala), arginine (Arg), asparagine (Asn), aspartic acid (Asp), cysteine (Cys), glutamine (Gln), glutamic acid (Glu), glycine (Gly), histidine (His), isoleucine (Ile), leucine (Leu), lysine (Lys), methionine (Met), phenylalanine (Phe), proline (Pro), serine (Ser), threonine (Thr), tryptophan (Trp), tyrosine (Tyr), and valine (Val); each of which may be L- or D- (where applicable). Furthermore, the term ‘amino acid’ may also include within its scope amino acid derivatives when such derivatives are not explicitly recited. Amino acid derivatives may be selected from those derivatized at the amino group or at the carboxy group or on the side chain. Preferred amino acid derivatives may include, but are not limited to, N-alkyl amino acids such as N-methylglycine otherwise known as sarcosine (Sar), N(α)-methylarginine (NMA), parachlorophenylalanine (p-Cl-Phe) and paranitrophenylalanine (p-NO₂-Phe) as well as N-acetyl amino acids. The phrase “amino acid or derivative thereof” also includes within its scope particular amino acid derivates discussed above and below.

Each incidence of the term “amino acid” within the present description and claims can therefore be considered to be interchangeable with the term “amino acid or derivative thereof”.

The term “tyrosine or a derivative thereof” (for example at X₁ in compounds of formula (I)) includes, for example,

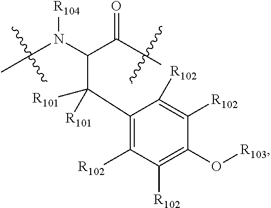

wherein each R₁₀₁ is independently selected from the group consisting of hydrogen, alkyl (especially C₁₋₆ alkyl; more especially methyl or ethyl) and halo (especially fluoro or chloro); (in one embodiment each R₁₀₁ is especially hydrogen);
wherein each R₁₀₂ is independently selected from the group consisting of hydrogen, alkyl (especially C₁₋₆ alkyl; more especially methyl or ethyl), halo (especially fluoro or chloro), nitro, —OH and —O-alkyl (especially —O—C₁₋₆ alkyl; more especially —O—CH₃ or —O—CH₂—CH₃); (in one embodiment each R₁₀₂ is especially independently selected from the group consisting of hydrogen, alkyl (especially C₁₋₆ alkyl; more especially methyl or ethyl), and halo (especially fluoro or chloro));
wherein R₁₀₃ is selected from the group consisting of hydrogen or alkyl (especially C₁₋₆ alkyl; more especially methyl or ethyl); (in one embodiment R₁₀₃ is especially hydrogen); and
wherein R₁₀₄ is selected from the group consisting of hydrogen or alkyl (especially C₁₋₆ alkyl; more especially methyl or ethyl); (in one embodiment R₁₀₄ is especially hydrogen).

The term “tyrosine or a derivative thereof” may refer to a L-derivative and/or a D-derivative.

The term “glycine or a derivative thereof” (for example at X₂ and/or X₃ in compounds of formula (I)) includes, for example,

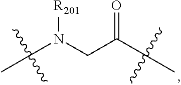

wherein R₂₀₁ is selected from the group consisting of hydrogen or alkyl (especially C₁₋₆ alkyl; more especially methyl or ethyl).

The term “phenylalanine or a derivative thereof” (for example at X₄ in compounds of formula (I)) includes, for example,

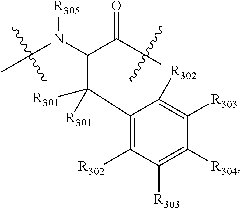

wherein each R₃₀₁ is independently selected from the group consisting of hydrogen, alkyl (especially C₁₋₆ alkyl; more especially methyl or ethyl) and halo (especially fluoro or chloro); (in one embodiment R₃₀₁ is especially hydrogen);
wherein each R₃₀₂ is independently selected from the group consisting of hydrogen, alkyl (especially C₁₋₆ alkyl; more especially methyl or ethyl), halo (especially fluoro or chloro), nitro, —OH or —O-alkyl (especially —O—C₁₋₆ alkyl; more especially —O—CH₃ or —O—CH₂—CH₃); (in one embodiment R₃₀₂ is especially independently selected from the group consisting of hydrogen, halo (especially fluoro or chloro), and nitro);
wherein each R₃₃ is independently selected from the group consisting of hydrogen, alkyl (especially C₁₋₆ alkyl; more especially methyl or ethyl), halo (especially fluoro or chloro), nitro, —OH or —O-alkyl (especially —O—C₁₋₆ alkyl; more especially —O—CH₃ or —O—CH₂—CH₃); (in one embodiment R₃₃ is especially independently selected from the group consisting of hydrogen, halo (especially fluoro or chloro), and nitro); and
wherein R₃₀₄ is selected from the group consisting of hydrogen, alkyl (especially C₁₋₆ alkyl; more especially methyl or ethyl), halo (especially fluoro or chloro), nitro, —OH or —O-alkyl (especially —O—C₁₋₆ alkyl; more especially —O—CH₃ or —O—CH₂—CH₃); (in one embodiment R₃₀₄ is especially independently selected from the group consisting of hydrogen, halo (especially fluoro or chloro), and nitro); and
wherein R₃₀₅ is selected from the group consisting of hydrogen or alkyl (especially C₁₋₆ alkyl; more especially methyl or ethyl); (in one embodiment R₃₀₅ is especially hydrogen).

The term “phenylalanine or a derivative thereof” may refer to a L-derivative and/or a D-derivative.

The term “leucine or a derivative thereof” (for example at X₅ in compounds of formula (I)) includes, for example,

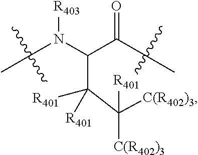

wherein each R₄₀₁ is independently selected from the group consisting of hydrogen, halo (especially fluoro or chloro), and cycloalkyl (especially cyclopentyl, cyclohexyl or cycloheptyl); (in one embodiment each R₄₀₁ is especially hydrogen or halo (especially fluoro or chloro); more especially each R₄₀₁ is hydrogen);
wherein each R₄₀₂ is independently selected from the group consisting of hydrogen, halo (especially fluoro or chloro) and cycloalkyl (especially cyclopentyl, cyclohexyl or cycloheptyl); (in one embodiment each R₄₀₂ is especially hydrogen or halo (especially fluoro or chloro); more especially each R₄₀₂ is hydrogen); and
wherein R₄₀₃ is selected from the group consisting of hydrogen or alkyl (especially C₁₋₆ alkyl; more especially methyl or ethyl); (in one embodiment R₄₀₃ is especially hydrogen);
wherein at least two groups selected from two R₄₀₁ groups, two R₄₀₂ groups, or one R₄₀₁ group and one R₄₀₂ group may together form a cycloalkyl (especially cyclopentyl, cyclohexyl or cycloheptyl).

The term “leucine or a derivative thereof” may refer to a L-derivative and/or a D-derivative.

The term “isoleucine or a derivative thereof” (for example at X₅ or X₈ in compounds of formula (I)) includes, for example,

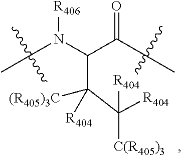

wherein each R₄₀₄ is independently selected from the group consisting of hydrogen, halo (especially fluoro or chloro), and cycloalkyl (especially cyclopentyl, cyclohexyl or cycloheptyl); (in one embodiment each R₄₀₄ is especially hydrogen or halo (especially fluoro or chloro); more especially each R₄₀₄ is hydrogen);
wherein each R₄₀₅ is independently selected from the group consisting of hydrogen, halo (especially fluoro or chloro) and cycloalkyl (especially cyclopentyl, cyclohexyl or cycloheptyl); (in one embodiment each R₄₀₅ is especially hydrogen or halo (especially fluoro or chloro); more especially each R₄₀₅ is hydrogen); and
wherein R₄₀₆ is selected from the group consisting of hydrogen or alkyl (especially C₁₋₆ alkyl; more especially methyl or ethyl); (in one embodiment R₄₀₆ is especially hydrogen);
wherein at least two groups selected from two R₄₀₄ groups, two R₄₀₅ groups, or one R₄₀₄ group and one R₄₀₅ group may together form a cycloalkyl (especially cyclopentyl, cyclohexyl or cycloheptyl).

The term “isoleucine or a derivative thereof” may refer to a L-derivative and/or a D-derivative.

The term “valine or a derivative thereof” (for example at X₅ or X₈ in compounds of formula (I)) includes, for example,

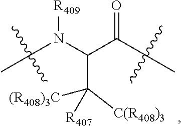

wherein R₄₀₇ is selected from the group consisting of hydrogen, halo (especially fluoro or chloro) and cycloalkyl (especially cyclopentyl, cyclohexyl or cycloheptyl); (in one embodiment R₄₀₇ is especially hydrogen or halo (especially fluoro or chloro); more especially R₄₀₇ is hydrogen) wherein each R₄₀₈ is independently selected from the group consisting of hydrogen, halo (especially fluoro or chloro) and cycloalkyl (especially cyclopentyl, cyclohexyl or cycloheptyl); (in one embodiment each R₄₀₈ is especially hydrogen or halo (especially fluoro or chloro); more especially each R₄₀₈ is hydrogen); and
wherein R₄₀₉ is selected from the group consisting of hydrogen or alkyl (especially C₁₋₆ alkyl; more especially methyl or ethyl); (in one embodiment R₄₀₉ is especially hydrogen);
wherein at least two groups selected from two R₄₀₈ groups, or one R₄₀₈ group and one R₄₀₇ group may together form a cycloalkyl (especially cyclopentyl, cyclohexyl or cycloheptyl).

The term “valine or a derivative thereof” may refer to a L-derivative and/or a D-derivative.

The term “arginine or a derivative thereof” (for example at X₆ and/or X₇ in compounds of formula (I)) includes, for example,

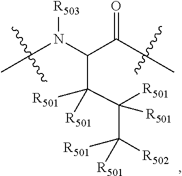

wherein each R₅₀₁ is independently selected from the group consisting of hydrogen, and halo (especially fluoro or chloro); (in one embodiment each R₅₀₁ is especially hydrogen);
wherein R₅₀₂ is selected from the group consisting of —NH—C(═NH)—NH2, or a 5- or 6-membered heterocyclic ring including one or more nitrogen atoms, wherein said heterocyclic ring may be substituted with one or more groups independently selected from the group consisting of hydrogen, alkyl (especially C₁₋₆ alkyl; more especially methyl or ethyl), halo (especially fluoro or chloro), nitro, —OH or —O-alkyl (especially —O—C₁₋₆ alkyl; more especially —O—CH₃ or —O—CH₂—CH₃); (in one embodiment R₅₀₂ is especially —NH—C(═NH)—NH2); and
wherein R₅₀₃ is selected from the group consisting of hydrogen or alkyl (especially C₁₋₆ alkyl; more especially methyl or ethyl).

When R₅₀₂ is a 5- or 6-membered heterocyclic ring including one or more nitrogen atoms, it may be monocyclic or bicyclic, and it may be aromatic or non-aromatic. Exemplary monocyclic 5- or 6-membered rings including one or more nitrogen atoms include, for example, pyridinyl, pyridazinyl, pyrimidinyl, pyrazinyl, 1,2,3-triazinyl, 1,2,4-triazinyl, 1,3,5-triazinyl, 1,2,3,4-tetrazinyl, 1,2,3,5-tetrazinyl, 1,2,4,5-tetrazinyl, pentazinyl, hexazinyl, pyrrolyl, pyrazolyl, imidazolyl, 1,2,3-triazolyl, 1,2,4-triazolyl, 1,2,5-triazolyl, 1,3,4-triazolyl, 1,2,3,4-tetrazolyl, 1,2,3,5-tetrazolyl, tetrahydropyrrolyl, tetrahydropyrazolyl, tetrahydroimidazolyl, tetrahydro-1,2,3-triazolyl, tetrahydro-1,2,4-triazolyl, tetrahydro-1,2,5-triazolyl, tetrahydro-1,3,4-triazolyl, tetrahydro-1,2,3,4-tetrazolyl, tetrahydro-1,2,3,5-tetrazolyl, piperidinyl, 1-4-diazacyclohexyl, 1,2-diazacyclohexyl, 1,3-diazacyclohexyl, 1,2,3-triazacyclohexyl, 1,2,4-triazacyclohexyl, 1,2,5-triazacyclohexyl, 1,2,6-triazacyclohexyl, 1,3,5-triazacyclohexyl, and tetrazacyclohexyl.

The term “arginine or a derivative thereof” may refer to a L-derivative and/or a D-derivative.

The term “proline or a derivative thereof” (for example at X₁₀ in compounds of formula (I)) includes, for example,

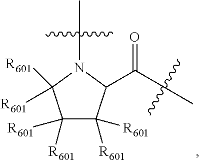

wherein R₆₀₁ is selected from the group consisting of hydrogen, and halo (especially fluoro or chloro); (in one embodiment R₆₀₁ is especially hydrogen).

The term “proline or a derivative thereof” may refer to a L-derivative and/or a D-derivative.

The term “alanine or a derivative thereof” (for example at X₈ in compounds of formula (I)) includes, for example,

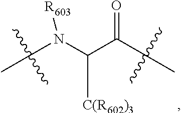

wherein each R₆₀₂ is independently selected from the group consisting of hydrogen, and halo (especially fluoro or chloro); (in one embodiment each R₆₀₂ is especially hydrogen); and
wherein R₆₀₃ is selected from the group consisting of hydrogen or alkyl (especially C₁₋₆ alkyl; more especially methyl or ethyl); (in one embodiment R₆₀₃ is especially hydrogen).

The term “alanine or a derivative thereof” may refer to a L-derivative and/or a D-derivative.

The term “lysine or a derivative thereof” (for example at X₁₁ in compounds of formula (I)) includes, for example,

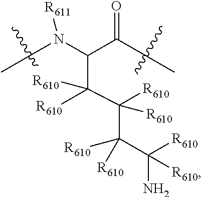

wherein each R₆₁₀ is independently selected from the group consisting of hydrogen, and halo (especially fluoro or chloro); (in one embodiment each R₆₁₀ is especially hydrogen); and
wherein R₆₁₁ is selected from the group consisting of hydrogen or alkyl (especially C₁₋₆ alkyl; more especially methyl or ethyl); (in one embodiment R₆₁₁ is especially hydrogen).

The term “lysine or a derivative thereof” may refer to a L-derivative and/or a D-derivative.

The term “alkyl” refers to a straight-chain or branched alkyl substituent containing from, for example, 1 to about 18 carbon atoms, preferably 1 to about 10 carbon atoms, more preferably 1 to about 8 carbon atoms, even more preferably from 1 to about 6 carbon atoms, still yet more preferably from 1 to 2 carbon atoms. Examples of such substituents include methyl, ethyl, propyl, isopropyl, n-butyl, sec-butyl, isobutyl, tert-butyl, pentyl, isoamyl, 2-methylbutyl, 3-methylbutyl, hexyl, heptyl, 2-methylpentyl, 3-methylpentyl, 4-methylpentyl, 2-ethylbutyl, 3-ethylbutyl, octyl, nonyl, decyl, undecyl, dodecyl and the like. The number of carbons referred to relate to the carbon backbone and carbon branching but does not include carbon atoms belonging to any substituents, for example the carbon atoms of an alkoxy substituent branching off the main carbon chain. Substituted alkyl includes alkyl substituted with one or more moieties selected from the group consisting of halo (e.g., Cl, F, Br, and I); other alkyl groups, halogenated alkyl (e.g., CF₃, 2-Br-ethyl, CH₂F, CH₂C1, CH₂CF₃, or CF₂CF₃); hydroxyl; amino; carboxylate; carboxamido; alkylamino; arylamino; guanidino; alkoxy; aryloxy; nitro; cyano; thio; sulfonic acid; sulfate; phosphonic acid; phosphate; and phosphonate as well as those described under the definition of ‘substituted’.

The term “amino” or “amine” as used herein means a moiety represented by the structure —NH₂, —NHR₁, —NR₁R₂, and N⁺R₁R₂R₃, includes primary, secondary, tertiary and quaternary amines/ammonium substituted by alkyl (i.e., alkylamino). Examples of such substituents (R₁-R₃) include hydrogen, alkyl, alkenyl, alkoxy, aryl, cycloalkyl, cycloalkenyl, heterocyclyl, and heteroaryl.

Whenever a range of the number of atoms in a structure is indicated (e.g., a C₁-C₁₂, C₁-C₁₀, C₁-C₉, C₁-C₆, C₁-C₄, alkyl, etc.), it is specifically contemplated that any sub-range or individual number of carbon atoms falling within the indicated range also can be used. Thus, for instance, the recitation of a range of 1-12 carbon atoms (e.g., C₁-C₁₂), 1-9 carbon atoms (e.g., C1-C), 1-6 carbon atoms (e.g., C₁-C₆), 1-4 carbon atoms (e.g., C₁-C₄), 1-3 carbon atoms (e.g., C₁-C₃), or 2-8 carbon atoms (e.g., C₂-C₈) as used with respect to any chemical group (e.g., alkyl, etc.) referenced herein encompasses and specifically describes 1, 2, 3, 4, 5, 6, 7, 8, 9, 10, 11, and/or 12 carbon atoms, as appropriate, as well as any sub-range thereof (e.g., 1-2 carbon atoms, 1-3 carbon atoms, 1-4 carbon atoms, 1-5 carbon atoms, 1-6 carbon atoms, 1-7 carbon atoms, 1-8 carbon atoms, 1-9 carbon atoms, 1-10 carbon atoms, 1-11 carbon atoms, 1-12 carbon atoms, 2-3 carbon atoms, 2-4 carbon atoms, 2-5 carbon atoms, 2-6 carbon atoms, 2-7 carbon atoms, 2-8 carbon atoms, 2-9 carbon atoms, 2-10 carbon atoms, 2-11 carbon atoms, 2-12 carbon atoms, 3-4 carbon atoms, 3-5 carbon atoms, 3-6 carbon atoms, 3-7 carbon atoms, 3-8 carbon atoms, 3-9 carbon atoms, 3-10 carbon atoms, 3-11 carbon atoms, 3-12 carbon atoms, 4-5 carbon atoms, 4-6 carbon atoms, 4-7 carbon atoms, 4-8 carbon atoms, 4-9 carbon atoms, 4-10 carbon atoms, 4-11 carbon atoms, and/or 4-12 carbon atoms, etc., as appropriate).

The term “substituted” in each incidence of its use herein, and in the absence of an explicit listing for any particular moiety, refers to substitution of the relevant moiety, for example an alkyl chain or ring structure, with one or more groups selected from C₁-C₁₂ alkyl, C₂-C₁₂ alkenyl, C₁-C₁₂ haloalkyl, C₁-C₁₂ alkoxy, CN, OH, SH, SeH, S-alkyl, oxo, NO₂, NH₂, NH—C(═NH)—NH₂, —NH—C(═NH)—NH—NO₂; —NH—C(═NH)-Me; —NH—SO₂-Me; —NH—C(═O)Me; monoalkyl ammonium, dialkyl ammonium, trialkylammonium, tetraalkylammonium, —NH—C(═NH)—NHMe; —NH—C(═NMe)-NHMe; —NH—C(═NH)—N(Me)₂; —NH—C(═NH)—NHCN; —NH—C(═O)—NH₂; —NH—C(═NH)—NH—OMe; —NH—C(═NH)—NHOH; (CH₂)₂—O—NH—C(═NH)—NH₂; (CH₂)₃—ONH₂, N(R₁)—C(═N₂)—N(R₃R₄) (R₁-R₄═H, alkyl) Cl, F, Br, I, COOH, cycloalkyl, imine, amide, aryl and heterocyclyl, each of which may themselves be optionally substituted. Furthermore, when any substituent is present, each substituent may be substituted with moieties that are independently selected from the group consisting of: halogen (e.g. chlorine, fluorine, bromine or iodine), ═O, ═S, —CN, —NO₂, —CF₃, —OCF₃, alkyl, alkenyl, haloalkyl, haloalkenyl, heteroalkyl, cycloalkyl, cycloalkenyl, heterocycloalkyl, heterocycloalkenyl, aryl, heteroaryl, cycloalkylalkyl, heterocyclo-alkylalkyl, heteroarylalkyl, arylalkyl, cycloalkylalkenyl, heterocycloalkylalkenyl, arylalkenyl, heteroarylalkenyl, cycloalkylheteroalkyl, heterocycloalkylheteroalkyl, arylheteroalkyl, heteroarylheteroalkyl, hydroxy, hydroxyalkyl, alkyloxy, alkyloxyalkyl, alkyloxycycloalkyl, alkyloxyheterocycloalkyl, alkyloxyaryl, alkyloxyheteroaryl, alkyloxycarbonyl, alkylaminocarbonyl, alkenyloxy, cycloalkyloxy, cycloalkenyloxy, heterocycloalkyloxy, heterocycloalkenyloxy, aryloxy, phenoxy, benzyloxy, heteroaryloxy, arylalkyloxy, amino, alkylamino, acylamino, aminoalkyl, arylamino, sulfonylamino, sulfinylamino, sulfonyl, alkylsulfonyl, arylsulfonyl, aminosulfonyl, sulfinyl, alkylsulfinyl, arylsulfinyl, aminosulfinylaminoalkyl, —C(═O)OH, —C(═O)Rₐ, C(═O)ORₐ, C(═O)NRₐR_{b}, C(═NOH)Rₐ, C(═NRₐ)NR_{b}R_{c}, NRₐR_{b}, NRₐC(═O)R_{b}, NRₐC(═O)OR_{b}, NRₐC(═O)NR_{b}R_{c}, NRₐC(═NR_{b})NR_{c}R_{d}, NRₐSO₂R_{b}, —SRₐ, SO₂NRₐR_{b}, —ORₐ, OC(═O)NRₐR_{b}, OC(═O)Rₐ and acyl, wherein Rₐ, R_{b}, R_{c} and R_{d} are each independently selected from the group consisting of H, C₁-C₁₂ alkyl, C₁-C₁₂ haloalkyl, C₂-C₁₂ alkenyl, C₁-C₁₀ heteroalkyl, C₃-C₁₂ cycloalkyl, C₃-C₁₂ cycloalkenyl, C₁-C₁₂ heterocycloalkyl, C₁-C₁₂ heterocycloalkenyl, C₆-C₁₈aryl, C₁-C₁₈ heteroaryl, and acyl, or any two or more of Rₐ, R_{b}, R_{c} and R_{d}, when taken together with the atoms to which they are attached form a heterocyclic ring system with 3 to 12 ring atoms.

The term “pharmaceutically acceptable salt” may include, for example, salts of the compounds of the invention with one or more alkali metal ions (for example, sodium, potassium), and/or with one or more alkaline earth metal ions (for example, magnesium or calcium).

The term “prodrug” is used in its broadest sense and encompasses those derivatives that are converted in vivo into the compounds of the invention. A prodrug may include modifications to one or more of the functional groups of a compound of the invention. The phrase “a derivative which is capable of being converted in vivo” as used in relation to another functional group includes all those functional groups of derivatives which upon administration into a mammal may be converted into the stated functional group. Those skilled in the art may readily determine whether a group may be capable of being converted in vivo to another functional group using routine enzymatic or animal studies. In some forms, prodrugs may include lipids, esters or ethers of compounds of the present invention.

Unless defined otherwise, all technical and scientific terms used herein have the same meaning as would be commonly understood by those of ordinary skill in the art to which this invention belongs.

Dynorphins are a class of opioid peptides. Dynorphins act primarily through the K-opioid receptor (KOP), a G-protein-coupled receptor. However, dynorphins also have affinity for the μ-opioid receptor (MOP) and 5-opioid receptor (DOP). As mentioned previously, it would be advantageous to provide for compounds that have improved selective activity to alleviate the problem of side-effects. The present invention is predicated, at least in part, on the finding that certain cyclic peptides have advantageous properties such as selective activity at selected receptor(s) and/or being less susceptible to metabolic degradation and/or treating pain when administered to a subject.

For ease of description, the peptides discussed herein have been generally described as amino acid sequences. These sequences are described without specifically showing the peptide bond formed between the amino acids. The person skilled in the art will appreciate that the peptides discussed in this manner have peptide bonds (namely, —CO—NH—) formed between adjacent amino acids. The peptide bonds are formed between the C-terminus of one amino acid and the N-terminus of the adjacent amino acid.

In a first aspect, although it need not be the only or indeed the broadest aspect, the invention resides in a compound of formula (I), or a salt or stereoisomer or prodrug or solvate thereof:

X₁-X₂-X₃-X₄-X₅-X₆-X₇-X₈-X₉-X₁₀-X₁₁ Formula (I)

wherein X₁, X₃, X₄, X₅, X₆ and X₇ are each independently an amino acid or derivative thereof; wherein X₂, X₈, X₉, X₁₀ and X₁₁, when present, are each independently an amino acid or derivative thereof; and

wherein a pair of any of X₁, X₂, X₃, X₄, X₅, X₆, X₇, X₈, X₉, X₁₀ and X₁₁ together form a linker

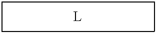

comprising

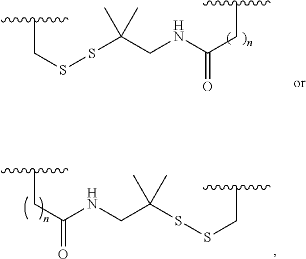

wherein n is 1 or 2.

In one embodiment,

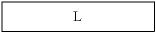

is formed between X₂ or X₃ and any remaining amino acid.

In an embodiment,

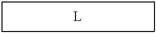

is formed between X₂ and X₅.

In an alternative embodiment,

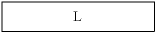

is formed between X₃ and X₅.

In another embodiment,

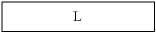

is formed between X₈ and X₁₀.

In another embodiment,

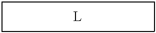

is formed between X₈ and X₉.

In one embodiment, X₁₁ is not present.

In one embodiment, X₁₀ and X₁₁ are not present.

In an embodiment, X₉, X₁₀ and X₁₁ are not present.

In another embodiment, X₈, X₉, X₁₀ and X₁₁ are not present.

In another embodiment, X₂ is not present.

In yet another embodiment, the invention relates to a compound of formula (II), or a salt or stereoisomer or solvate or prodrug thereof:

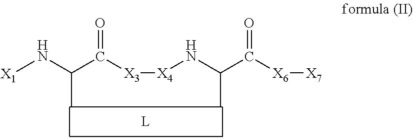

wherein X₁, X₃, X₄, X₆ and X₇ are each independently an amino acid or derivative thereof;
wherein

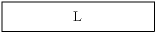

comprises

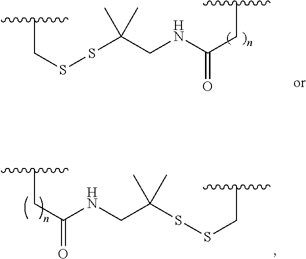

wherein n is 1 or 2.

In another form, the invention resides in a compound of formula (III), or a salt or stereoisomer or solvate or prodrug thereof:

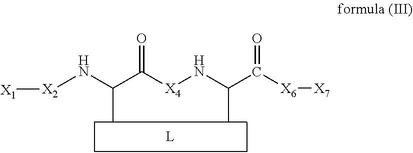

wherein X₁, X₂, X₄, X₆ and X₇ are each independently an amino acid or derivative thereof; and

wherein

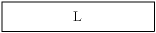

comprises

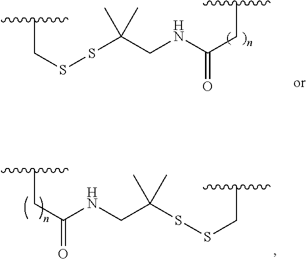

wherein n is 1 or 2.

In one form, the invention resides in a compound of formula (IV), or a salt or stereoisomer or solvate or prodrug thereof:

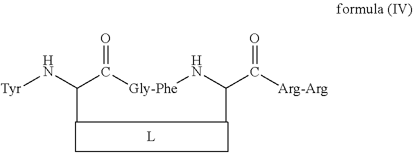

wherein

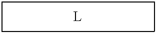

comprises

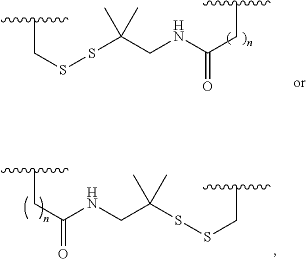

wherein n is 1 or 2.

In another form, the invention resides in a compound of formula (V), or a salt or stereoisomer or solvate or prodrug thereof:

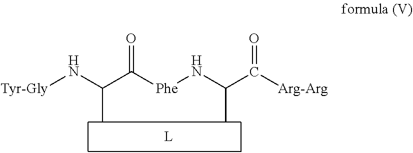

wherein

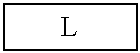

comprises

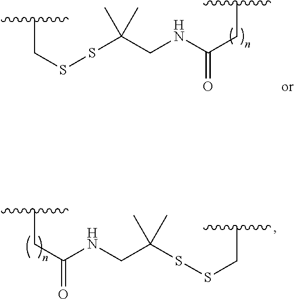

wherein n is 1 or 2.

In another embodiment, the invention relates to a compound of formula (IX), or a salt or stereoisomer or solvate or prodrug thereof:

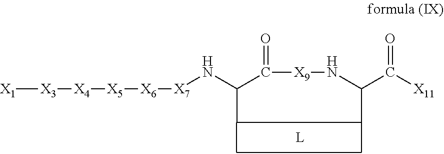

wherein X₁, X₃, X₄, X₅, X₆, X₇, X₉ and X₁₁ are each independently an amino acid or derivative thereof;
wherein

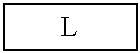

comprises

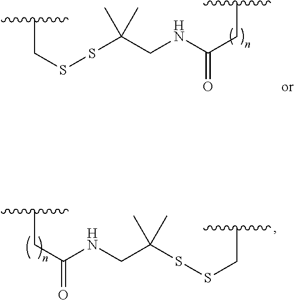

wherein n is 1 or 2.

In one embodiment of the compounds of formula (I), (II), (III), (IV), (V) or (IX), where applicable: X₁ is tyrosine or a derivative thereof; X₄ is phenylalanine or a derivative thereof; and X₆ is arginine or a derivative thereof.

In one embodiment, of the compounds of formula (I), (II), (III), (IV), (V) or (IX), where applicable:
X₁ is tyrosine or a derivative thereof;
X₄ is phenylalanine or a derivative thereof;
If X₅ does not form part of the linker

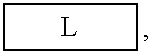

X₅ is selected from the group consisting of: leucine or a derivative thereof, isoleucine or a derivative thereof, and valine or a derivative thereof;
X₆ is arginine or a derivative thereof; and
If X₇ does not form part of the linker

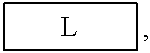

X₇ is arginine or a derivative thereof.

In one embodiment, of the compounds of formula (I), (II), (III), (IV), (V) or (IX), where applicable:
X₁ is tyrosine or a derivative thereof;
X₄ is phenylalanine or a derivative thereof;
If X₅ does not form part of the linker

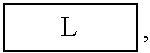

X₅ is selected from the group consisting of: leucine or a derivative thereof, isoleucine or a derivative thereof, and valine or a derivative thereof;
X₆ is arginine or a derivative thereof; and
X₇ is arginine or a derivative thereof.

In one embodiment, of the compounds of formula (I), (II), (III), (IV), (V) or (IX), where applicable:
If X₁ does not form part of the linker

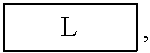

X₁ is tyrosine or a derivative thereof;
If X₄ does not form part of the linker

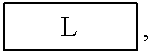

X₄ is phenylalanine or a derivative thereof; and
If X₆ does not form part of the linker

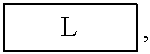

X₆ is arginine or a derivative thereof.

In one embodiment of the compounds of formula (I), (II), (III), (IV), (V) or (IX), where applicable: X₁ is tyrosine or a derivative thereof; X₄ is phenylalanine or a derivative thereof; X₅ is selected from the group consisting of: leucine or a derivative thereof, isoleucine or a derivative thereof, and valine or a derivative thereof; X₆ is arginine or a derivative thereof; and X₇ is arginine or a derivative thereof.

In one embodiment, of the compounds of formula (I), (II), (III), (IV), (V) or (IX), where applicable:
If X₁ does not form part of the linker

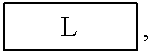

X₁ is tyrosine or a derivative thereof;
If X₄ does not form part of the linker

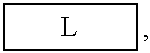

X₄ is phenylalanine or a derivative thereof;
If X₅ does not form part of the linker

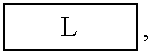

X₅ is selected from the group consisting of: leucine or a derivative thereof, isoleucine or a derivative thereof, and valine or a derivative thereof;
If X₆ does not form part of the linker

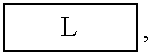

X₆ is arginine or a derivative thereof; and
If X₇ does not form part of the linker

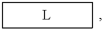

X₇ is arginine or a derivative thereof.

In embodiments of the compounds of formula (I), (II), (III), (IV), (V) or (IX) one or more of the following may apply:
n may be 1.
n may be 2.

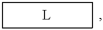

may comprise

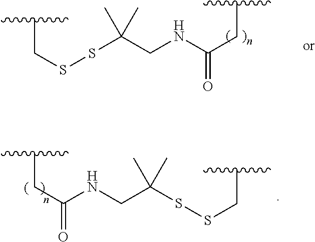

where applicable, X₁, X₂, X₃, X₄, X₅, X₆, X₇, X₈, X₉, X₁₀ and X₁₁ may each independently be an L-amino acid or derivative thereof, or a D-amino acid or derivative thereof.
where applicable, X₁, X₂, X₃, X₄, X₅, X₆, X₇, X₈, X₉, X₁₀ and X₁₁ may each be independently selected the group consisting of Tyr, Gly, Phe, Leu, Arg, Ile, Pro, Sar, p-Cl-Phe, NMA, p-Cl-Phe, p-NO₂-Phe, Asp and Lys.
where applicable, X₁ may be tyrosine or a derivative thereof; especially tyrosine. In a further embodiment, where applicable, X₁ is L-Tyr. In another embodiment, where applicable, X₁ is D-Tyr.
where applicable, X₂ may be glycine or a derivative thereof; especially N-alkyl glycine; more especially sarcosine.
where applicable, X₂ may be absent.
where applicable, X₂ may be Gly.
where applicable, X₃ may be glycine or a derivative thereof.
where applicable, X₃ may be N-alkyl glycine (especially sarcosine).
where applicable, X₄ may be phenylalanine or a derivative thereof. In a further embodiment, where applicable, X₄ is L-Phe. In another embodiment, where applicable, X₄ is D-Phe.
where applicable, X₄ may be phenylalanine optionally substituted by one or more of halo (especially chloro or fluoro), or nitro; especially phenylalanine substituted by chloro or nitro. The phenylalanine may be substituted in any suitable position, especially on the phenyl group, more especially at a para position on the phenyl group. The optionally substituted phenylalanine may be optionally substituted L-phenylalanine.
where applicable, X₅ may be leucine or a derivative thereof. In a further embodiment, where applicable, X₅ is L-Leu. In another embodiment, where applicable, X₅ is D-Leu.
where applicable, X₆ may be arginine or a derivative thereof. In a further embodiment, where applicable, X₆ is L-Arg. In another embodiment, where applicable, X₆ is D-Arg. In another embodiment, X₆ may be N(α)-alkyl Arg. In a further embodiment, where applicable, X₆ is N(α)-alkyl L-Arg; especially N(α)-methyl L-Arg. In another embodiment, where applicable, X₆ is N(α)-alkyl D-Arg; especially N(α)-methyl D-Arg.
where applicable, X₇ may be arginine or a derivative thereof. In a further embodiment, where applicable, X₇ is L-Arg. In another embodiment, where applicable, X₇ is D-Arg. In another embodiment, X₇ may be N(α)-alkyl Arg. In a further embodiment, where applicable, X₇ is N(α)-alkyl L-Arg; especially N(α)-methyl L-Arg. In another embodiment, where applicable, X₇ is N(α)-alkyl D-Arg; especially N(α)-methyl D-Arg.
where applicable, X₈ may be isoleucine or a derivative thereof, leucine or a derivative thereof, valine or a derivative thereof, phenylalanine or a derivative thereof, alanine or a derivative thereof or may be absent; especially X₈ may be L-isoleucine; D-leucine, D-valine, D-phenylalanine, D-alanine or may be absent; more especially L-isoleucine. In another embodiment, X₈ may be Ile. In a further embodiment, where applicable, X₈ is L-Ile. In another embodiment, where applicable, X₈ is D-Ile.
where applicable, X₈ may be arginine or a derivative thereof. In a further embodiment, where applicable, X₉ is L-Arg. In another embodiment, where applicable, X₈ is D-Arg.
where applicable, X₁₀ may be proline or a derivative thereof. In a further embodiment, where applicable, X₁₀ is L-Pro. In another embodiment, where applicable, X₁₀ is D-Pro.
where applicable, X₁₁ may be lysine or a derivative thereof. In a further embodiment, where applicable, X₁₁ is L-Lys. In another embodiment, where applicable, X₁₁ is D-Lys.
where applicable, X₁ may be Tyr, and X₆ and X₇ independently may be Arg or N(α)-alkyl Arg. In a particularly preferred embodiment, where applicable, X₁ is L-Tyr, and X₆ and X₇ may be independently L-Arg, D-Arg, N(α)-methyl L-Arg, or N(α)-methyl D-Arg.

In preferred embodiments, the compound is selected from the group consisting of:

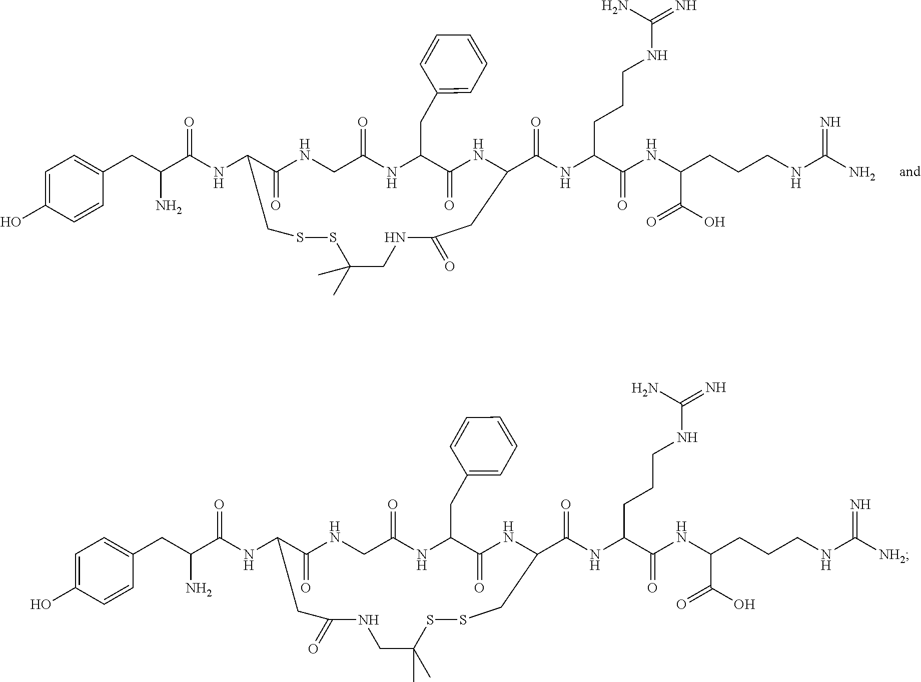 [SMILES: CC1(C)CNC(=O)CC(C(=O)NC(CCCNC(=N)N)C(=O)NC(CCCNC(=N)N)C(=O)O)NC(=O)C(Cc2ccccc2)NC(=O)CNC(=O)C(NC(=O)C(N)Cc2ccc(O)cc2)CSS1 ; CC1(C)CNC(=O)CC(NC(=O)C(N)Cc2ccc(O)cc2)C(=O)NCC(=O)NC(Cc2ccccc2)C(=O)NC(C(=O)NC(CCCNC(=N)N)C(=O)NC(CCCNC(=N)N)C(=O)O)CSS1]

or a salt or stereoisomer or solvate or prodrug thereof.

In certain embodiments, where X₁-X₁₁ are present,

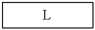

is formed between X₈ and any remaining amino acid or derivative thereof. In embodiments, where X₁-X₁₁ are present,

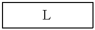

is formed between X₈ and X₁₀. In this embodiment, X₂ may be absent.

In certain embodiments, where X₁-X₉ are present,

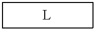

is formed between X₈ and any remaining amino acid or derivative thereof. In embodiments, where X₁-X₉ are present,

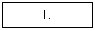

is formed between X₈ and X₉. In these embodiments, X₂ may be absent.

In some embodiments, X₂ or X₃ may be absent. In this regard, in the instance where X₂ is absent, X₁ and X₃ are bound. In the instance where X₃ is absent, X₂ and X₄ are bound.

It will be appreciated that the N-terminus of the compounds of the present invention may be unsubstituted (i.e. providing NH₂— or NH₃⁺—), or be acylated, for example with a C₁₋₆alkyl-CO group (i.e. providing C₁₋₆alkyl-CO—NH—). An exemplary acyl N-terminal group is acetyl.

It will be appreciated that the C-terminus of the compounds of the present invention may terminate in a COOH (or COO⁻) or CONH₂ moiety. In this regard, the use of a Rink amide resin during solid phase synthesis can lead to the formation of CONH₂ at the C-terminus. Further to this, the use of Wang resin during the synthesis can lead to the formation of the COOH at the C-terminus. In this regard, in some embodiments, the C-terminus of the compound of the first aspect is COOH or CONH₂. In an embodiment, the compound is selected from the group consisting of:

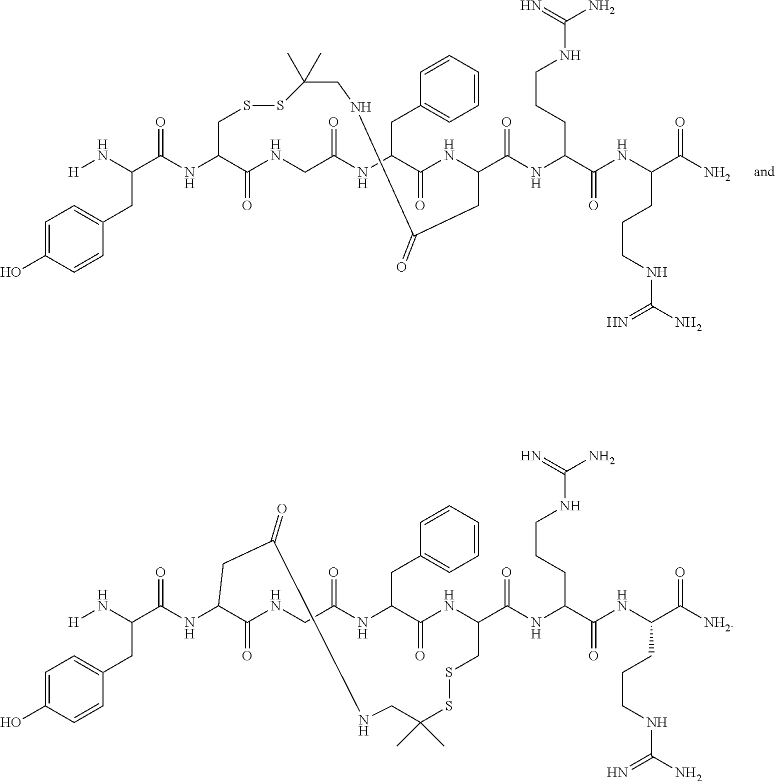 [SMILES: CC1(C)CNC(=O)CC(C(=O)NC(CCCNC(=N)N)C(=O)NC(CCCNC(=N)N)C(N)=O)NC(=O)C(Cc2ccccc2)NC(=O)CNC(=O)C(NC(=O)C(N)Cc2ccc(O)cc2)CSS1 ; CC1(C)CNC(=O)CC(NC(=O)C(N)Cc2ccc(O)cc2)C(=O)NCC(=O)NC(Cc2ccccc2)C(=O)NC(C(=O)NC(CCCNC(=N)N)C(=O)N[C@@H](CCCNC(=N)N)C(N)=O)CSS1]

In one embodiment, the compound is selected from the group consisting of:

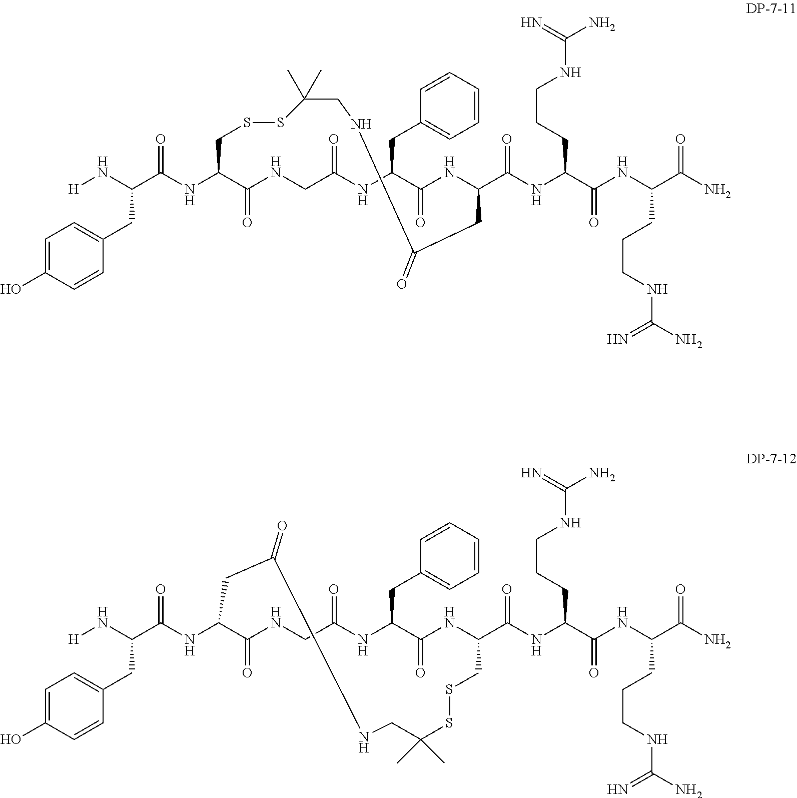 [SMILES: CC1(C)CNC(=O)C[C@H](C(=O)N[C@@H](CCCNC(=N)N)C(=O)N[C@@H](CCCNC(=N)N)C(N)=O)NC(=O)[C@H](Cc2ccccc2)NC(=O)CNC(=O)[C@@H](NC(=O)[C@@H](N)Cc2ccc(O)cc2)CSS1 ; CC1(C)CNC(=O)C[C@@H](NC(=O)[C@@H](N)Cc2ccc(O)cc2)C(=O)NCC(=O)N[C@@H](Cc2ccccc2)C(=O)N[C@H](C(=O)N[C@@H](CCCNC(=N)N)C(=O)N[C@@H](CCCNC(=N)N)C(N)=O)CSS1]

It is postulated that the Arg groups in the X₆ and X₇ position can be metabolized. In this regard, the inventors believe that incorporation of the X₆ and X₇ amino acids into the cyclic structure may improve the metabolic stability of the dynorphin analogue. The inventors also believe that use of D-arginine or N(α)-methyl arginine (especially N(α)-methyl L-arginine) at X₆ and/or X₇ may also improve metabolic stability. In one embodiment of the compound of formula (I),

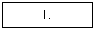

is formed between X₅ and any one of X₇, X₈, X₉, X₁₀ and X₁₁. In an embodiment of the compound of formula (I),

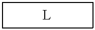

is formed between X₅ and X₇.

In another form, the invention resides in a compound of formula (VI), or a salt or stereoisomer or solvate or prodrug thereof:

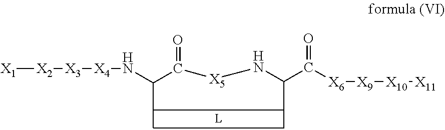

wherein X₁, X₂, X₃, X₄, X₆, are each independently an amino acid or derivative thereof;

wherein X₈, X₉, X₁₀ and X₁₁, when present, are each independently an amino acid or derivative thereof; and

wherein

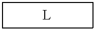

comprises 0 or

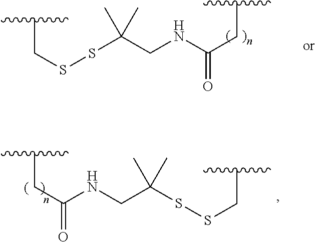

wherein n is 1 or 2.

In another form, the invention resides in a compound of formula (VII), or a salt or stereoisomer or solvate or prodrug thereof:

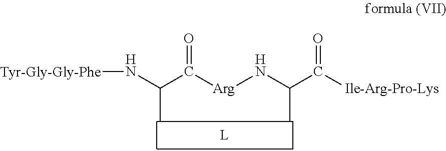

wherein

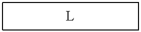

comprises

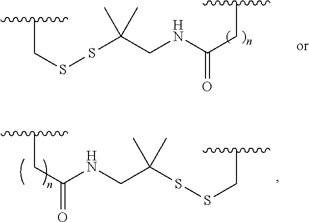

wherein n is 1 or 2.

It is postulated that linear dynorphins (e.g., dynorphin 1-17 and dynorphin 1-7) are metabolized quickly in vivo. These linear dynorphins can metabolize within a few minutes to a few seconds which is too short for them to function as a drug. In this regard, it is postulated that the incorporation of the dynorphin structure (e.g. DP-7-00 mentioned hereinafter) into a cyclic structure may improve the metabolic stability of the resulting compound. Furthermore, incorporation of a disulfide bond into the cyclic structure is believed to be advantageous because the disulfide bond can subsequently be cleaved within cells by thio-disulfide exchange to metabolize the cyclic structure thereby forming a linear structure. The gem-dimethyl group is also postulated to provide chemical and/or metabolic stability to the disulfide bond.

In regard to metabolic stability, this relates to the half-life or time it takes for the compound of the first aspect to metabolize in vivo. This can be tested using trypsin and serum stability studies. Compounds of the present invention may also have improved shelf-life stability, which relates to the compounds remaining within their product specification while stored under defined conditions.

Introduction of the disulfide bond during chemical synthesis remains a significant challenge due to the complex thiol-protection and deprotection strategies required and the base liability of the disulfide bond.

The disulfide bond is preferably a pre-generated component of the peptide which is provided with an amino group and a disulfide bond. A preferred amino acid building block is:

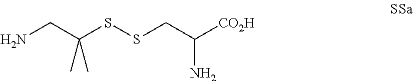

It will be appreciated that SSa can be protected or deprotected. Furthermore, SSa can be utilized to incorporate the disulfide bond into the peptide structure. The terminal amino group on the side chain can be used to form a linker structure

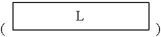

with a carboxylic group on a side chain of another amino acid in the molecule. For instance, the carboxylic group may be present as an aspartic acid in another part of the molecule. This allows for the disulfide bond to be incorporated into a cyclic structure. For instance, DP-7-11 can be formed by having SSa as X₂ and aspartic acid as X₅, and subsequently coupled to each other to form

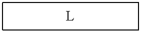

as

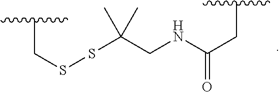 [SMILES: *CSSC(C)(C)CNC(=O)C*]

Similarly, DP-7-12 can be formed by having aspartic acid as X₂ and SSa as X₅, and subsequently coupled to each other to form

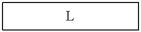

as

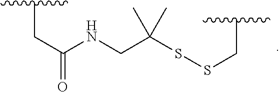 [SMILES: *CSSC(C)(C)CNC(=O)C*]

It will be appreciated that substitution of any two of X₁-X₁₁ with SSa and aspartic acid can lead to cyclization between any two of X₁-X₁₁. In one embodiment, one of X₁-X₁₁ is SSa. In an embodiment, one of X₁-X₁₁ is aspartic acid.

The SSa can be synthesized using solid phase peptide synthesis or solution phase peptide synthesis. The synthesis of SSa is discussed in PCT/AU2018/050773 and is incorporated herein by reference in its entirety.

It will be appreciated that n can be 1 by coupling SSa with aspartic acid (n=1). In another embodiment, n is 2 when SSa is coupled with glutamic acid (n=2). In some embodiments, any one of X₂, X₃, X₅, X₇, X₈, X₉, X₁₀ and X₁₁ is SSa, especially any one of X₂, X₃, X₅, X₈, X₉ and X₁₀ is SSa. In some embodiments, any one of X₂, X₃, X₅, X₇, X₈, X₉, X₁₀ and X₁₁ is aspartic acid or glutamic acid, especially any one of X₂, X₃, X₅, X₈, X₉ and X₁₀ is aspartic acid or glutamic acid.

It will be appreciated that

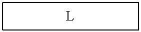

forms a cyclic structure with the any two of X₁-X₁₁ and the amino acids between said two of X₁-X₁₁.

Another advantage of the compounds of the present invention is that they can be synthesized relatively easily. In this regard, the person skilled in the art will appreciate that the compounds of the present invention are peptides that can be synthesized utilizing standard solid phase peptide synthesis or solution phase peptide synthesis protocols known in the art.

The present synthetic method allows for a large number of cyclic dynorphin-like compounds to be accessible due to the ease of modification through using different amino acids.

In a second aspect, the present invention relates to a compound of formula (I), or a salt or stereoisomer or solvate or prodrug thereof:

X₁-X₂-X₃-X₄-X₅-X₆-X₇-X₈-X₉-X₁₀-X₁₁ Formula (I)

wherein X₁, X₂, X₃, X₄, X₅, X₆ and X₇ are each independently an amino acid; wherein X₈, X₉, X₁₀ and X₁₁, when present, are each independently an amino acid; and

wherein a pair of any of X₁, X₂, X₃, X₄, X₅, X₆, X₇, X₈, X₉, X₁₀ and X₁₁ together form a linker

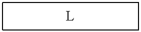

comprising

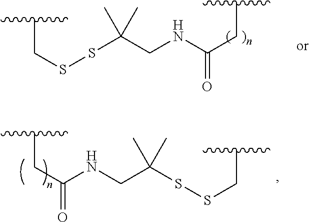

wherein n is 1 or 2.

Features of the second aspect of the present invention may be as described for the first aspect.

In a third aspect, the compound of the present invention can be viewed as a compound of formula (XI), or a salt or stereoisomer or solvate or prodrug thereof:

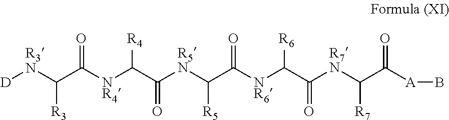

wherein, when present, A has structure

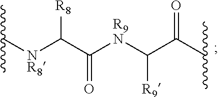

wherein, when present, B has a structure

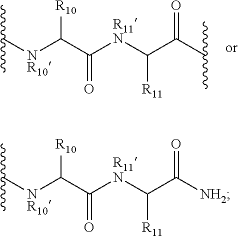

or A-B has the structure —OH or —NH₂;

wherein, D has a structure

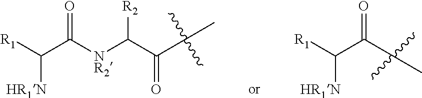

wherein R₁-R₁₁ and R₁′-R₁₁′ are independently selected from the group consisting of hydrogen, optionally substituted alkyl, optionally substituted alkyl-phenyl; and wherein a pair of any one of R¹-R¹¹ together form a linker

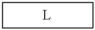

comprising

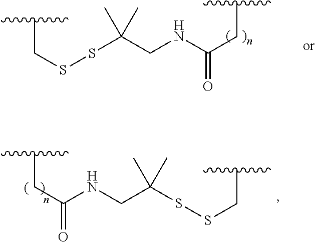

wherein n is 1 or 2, and wherein any pair of R₁ and R₁′, R₂ and R₂′, R₃ and R₃′, R₄ and R₄′, R₅ and R₅′, R₆ and R₆′, R₇ and R₇′, R₈ and R₈′, R₉ and R₉′, R₁₀ and R₁₀′, and R₁₁ and R₁₁′ may together form a cyclic structure.

In an alternative embodiment, the compound of the present invention can be viewed as a compound of formula (X), or a salt or stereoisomer or solvate or prodrug thereof:

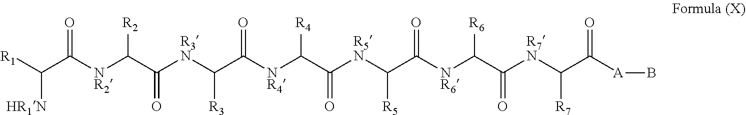

wherein, when present, A has structure

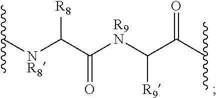

wherein, when present, B has a structure

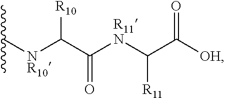

wherein R₁-R₁₁ and R₁′-R₁₁′ are independently selected from the group consisting of hydrogen, and substituted or unsubstituted alkyl; and wherein a pair of any one of R₁-R₁₁ together form a linker

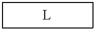

comprising

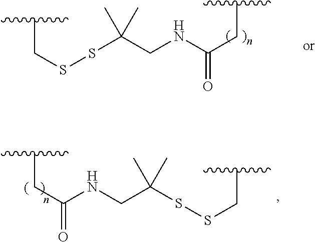

wherein n is 1 or 2, and wherein any pair of R₁ and R₁′, R₂ and R₂′, R₃ and R₃′, R₄ and R₄′, R₅ and R₅′, R₆ and R₆′, R₇ and R₇′, R₈ and R₈′, R₉ and R₉′, R₁₀ and R₁₀′, and R₁₁ and R₁₁′ may together form a cyclic structure.

In some embodiments of compounds of the formula (XI) or (X), one or more of the following may apply:
B may not be present.
A and B both may not be present.
A-B may be —OH or —NH₂;
D may be

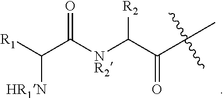

In another embodiment, D is

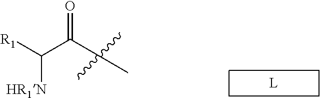

may be formed between either R₂ or R₃ and any remaining R groups. In one embodiment,

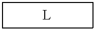

is formed between R₂ and R₅. In another embodiment,

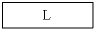

is formed between R₃ and R₅. In one embodiment,

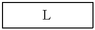

is formed between R₈ and R₁₀.
R₁′-R₁₁′ may be independently selected from hydrogen or C₁-C₆ alkyl; especially hydrogen, methyl or ethyl. In one embodiment, R₁′-R₁₁′ are all hydrogen. In another embodiment, R₁′-R₁₁′ are hydrogen or methyl.
R₁′-R₁₁′ may be independently selected from the group consisting of hydrogen and unsubstituted alkyl. In one embodiment, R₁′-R₁₁′ are independently selected from the group hydrogen and substituted or unsubstituted C₁-C₄ alkyl.
where applicable, R₁-R₁₁ may be independently selected from the group consisting of hydrogen, alkyl optionally substituted (with —COOH, —NH₂, —NH—C(═NH)—NH₂), and alkyl-phenyl (wherein the phenyl is optionally substituted with one or more of —OH, —Cl, —NO₂).
where applicable, R₁-R₁₁ may be independently selected from the group consisting of

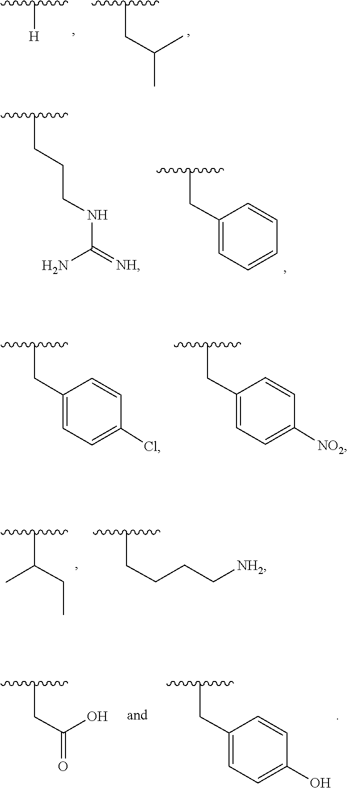

where applicable, R₁-R₇ may be independently selected from the group consisting of

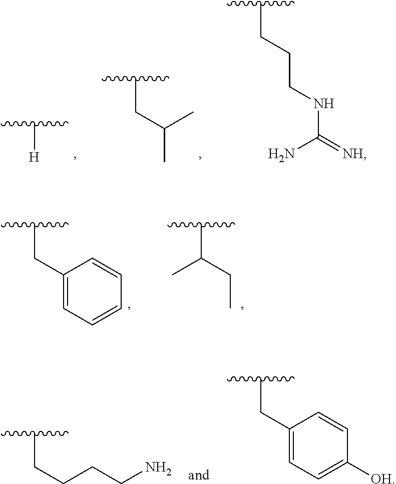

where applicable, one or more of R₁ and R₁′, R₂ and R₂′, R₃ and R₃′, R₄ and R₄′, R₅ and R₅′, R₆ and R₆′, R₇ and R₇′, R₈ and R₈′, R₉ and R₉′, R₁₀ and R₁₀′, and R₁₁ and R₁₁′ together form

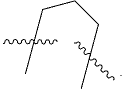 [SMILES: *CCC*]

where applicable, R₁ may be

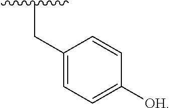 [SMILES: *Cc1ccc(O)cc1]

In an embodiment, where applicable, R₁ is

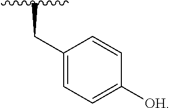

In an embodiment, where applicable, R₁ is

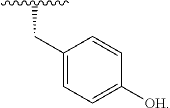

where applicable, R₂ may be

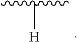 [SMILES: *[H]]

where applicable, may be

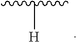 [SMILES: *[H]]

where applicable, R₄ may be

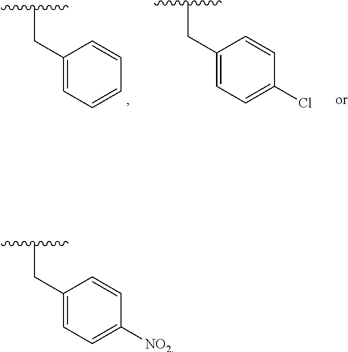 [SMILES: *Cc1ccccc1 ; *Cc1ccc(Cl)cc1 ; *Cc1ccc([N+](=O)[O-])cc1]

In certain embodiments, where applicable, R₄ is

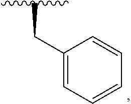

In one embodiment, where applicable, R₄ is

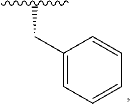

In one embodiment, where applicable, R₄ may be

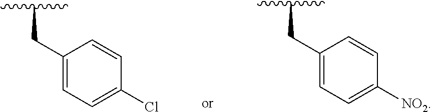

In one embodiment, where applicable, R₄ may be

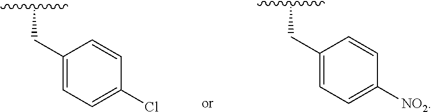

where applicable, R₅ may be

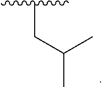 [SMILES: *CC(C)C]

In an embodiment, where applicable, R₅ is

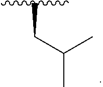

In certain embodiments, where applicable, R₅ is

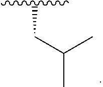

where applicable, R₆ may be

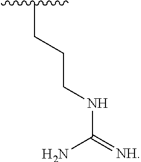 [SMILES: *CCCNC(=N)N]

In one embodiment, where applicable, R₆ is

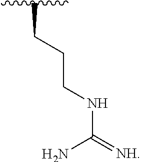

In one embodiment, where applicable, R₆ is

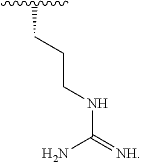

where applicable, R₇ may be

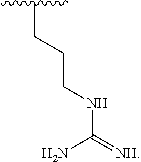 [SMILES: *CCCNC(=N)N]

In an embodiment, where applicable, R₇ is

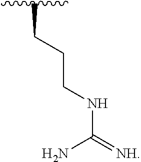

In one embodiment, where applicable, R₇ is

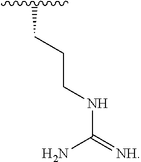

where applicable, R₈ may be

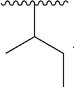 [SMILES: *C(C)CC]

In one embodiment where applicable, R₈ is

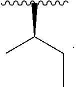 [SMILES: *[C@H](C)CC]

In one embodiment, where applicable, R₈ is

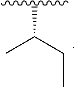 [SMILES: *[C@@H](C)CC]

where applicable, R₉ may be

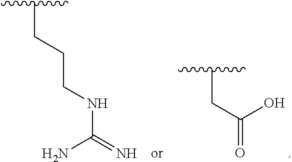 [SMILES: *CCCNC(=N)N ; *CC(=O)O]

In one embodiment, where applicable, R₉ is

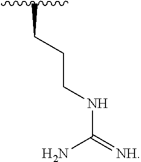

In one embodiment, where applicable, R₉ is

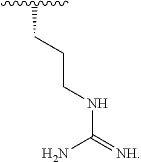

where applicable, R₁₀ and R₁₀′ may together form

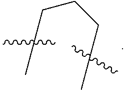 [SMILES: *CCC*]

where applicable, R₁₁ may be

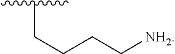 [SMILES: *CCCCN]

In one embodiment, where applicable, R₁₁ is

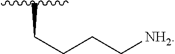

In one embodiment, where applicable, R₁₁ is

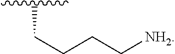

the salt may be a pharmaceutically acceptable salt.

For ease of description, the following embodiments of the compound of the first aspect are described in amino acid sequence. The following naming convention is used c([X1]-[X2]-[X3]-[X4]-[X5]-[X6]-[X7]-[X8]-[X9]-[X10]-[X11]), wherein the linker

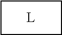

is formed between the SSa and Asp amino acids. It will be appreciated that in some of these embodiments, one or more of X2 and/or X8-X11 are not present.

In one embodiment, the compound of the present invention is selected from the group consisting of:

SEQ ID NO: 1
(DP-7-11) or (CP5) - c(Tyr-SSa-Gly-Phe-D(Asp)-Arg-
Arg)
SEQ ID NO: 2
(DP-7-12) or (CP6) - c(Tyr-D(Asp)-Gly-Phe-SSa-Arg-
Arg)
SEQ ID NO: 3
(DP-7-06a) - c(Tyr-SSa-Gly-D(Phe)-Asp-Arg-Arg)
SEQ ID NO: 4
(DP-7-07a) - c(Tyr-Asp-Gly-D(Phe)-SSa-Arg-Arg)
SEQ ID NO: 5
(DP-7-08a) - c(Tyr-Gly-SSa-D(Phe)-Asp-Arg-Arg)
SEQ ID NO: 6
(DP-7-09a) - c(Tyr-Gly-Asp-D(Phe)-SSa-Arg-Arg)
SEQ ID NO: 7
(DP-7-10) - c(Tyr-D(SSa)-Gly-Phe-D(Asp)-Arg-Arg)
SEQ ID NO: 8
(DP-7-11a) - c(Tyr-SSa-Gly-Phe-D(Asp)-D(Arg)-Arg)
SEQ ID NO: 9
(DP-7-11 b) - c(Tyr-SSa-Gly-Phe-D(Asp)-Arg-D(Arg))
SEQ ID NO: 10
(DP-7-11c) - c(Tyr-SSa-Gly-Phe-D(Asp)-D(Arg)-
D(Arg))
SEQ ID NO: 11
(DP-7-13) or (CP7) - c(Tyr-Gly-SSa-Phe-D(Asp)-Arg-
Arg)
SEQ ID NO: 12
(DP-7-14) or (CP8) - c(Tyr-Gly-D(Asp)-Phe-SSa-Arg-
Arg)
SEQ ID NO: 13
(DP-9-01a) - c(Tyr-Gly-Gly-Phe-D(Asp)-Arg-SSa-
D(Leu)-Arg)
SEQ ID NO: 14
(DP-9-01b) - c(Tyr-Gly-Gly-Phe-D(Asp)-Arg-SSa-
D(Val)-Arg)
SEQ ID NO: 15
(DP-9-01c) - c(Tyr-Gly-Gly-Phe-D(Asp)-Arg-SSa-
D(Phe)-Arg)
SEQ ID NO: 16
(DP-9-02a) - c(Tyr-Gly-Gly-Phe-SSa-Arg-D(Asp)-
D(Phe)-Arg)
SEQ ID NO: 17
(DP-9-03a) - c(Tyr-Gly-Gly-Phe-Leu-Arg-SSa-
D(Phe)-Asp)
SEQ ID NO: 18
(DP-9-03b) - c(Tyr-Gly-Gly-Phe-Leu-Arg-SSa-D(Val)-
Asp)
SEQ ID NO: 19
(DP-9-04a) - c(Tyr-SSa-Gly-Phe-Asp-Arg-Arg-D(Val)-
Arg)
SEQ ID NO: 20
(DP-11-01) - c(Tyr-Gly-Gly-Phe-Asp-Arg-SSa-Ile-Arg-
Pro-Lys)
SEQ ID NO: 21
(DP-11-02) - c(Tyr-Gly-Gly-Phe-Asp-Arg-Arg-SSa-Arg-
Pro-Lys)
SEQ ID NO: 22
(DP-11-03) - c(Tyr-Gly-Gly-Phe-Asp-Arg-Arg-Ile-SSa-
Pro-Lys)
SEQ ID NO: 23
(DP-11-04) - c(Tyr-Gly-Gly-Phe-Asp-Arg-Arg-Ile-Arg-
SSa-Lys)
SEQ ID NO: 24
(DP-11-05) - c(Tyr-Gly-Gly-Phe-Asp-Arg-Arg-Ile-Arg-
Pro-SSa)
SEQ ID NO: 25
(DP-11-06) - c(Tyr-Gly-Gly-Phe-D(Asp)-Arg-SSa-Ile-
Arg-Pro-Lys)
SEQ ID NO: 26
(DP-11-01a) - c(Tyr-Ala-Gly-Phe-Asp-Arg-SSa-Ile-
Arg-Pro-Lys)
SEQ ID NO: 27
(DP-11-01b) - c(Tyr-Gly-Ala-Phe-Asp-Arg-SSa-Ile-
Arg-Pro-Lys)
SEQ ID NO: 28
(DP-11-01c) - c(Tyr-Gly-D(Asp)-Phe-Asp-Arg-SSa-Ile-
Arg-Pro-Lys
SEQ ID NO: 29
(DP-11-01d) - c(Tyr-Gly-Gly-Trp-Asp-Arg-SSa-Ile-
Arg-Pro-Lys)
SEQ ID NO: 30
(DP-11-01e) - c(Tyr-Gly-Gly-Tyr-Asp-Arg-SSa-Ile-
Arg-Pro-Lys)
SEQ ID NO: 31
(DP-11-01f) - c(Tyr-Gly-Gly-Phe-Asp-Arg-SSa-Ala-
Arg-Pro-Lys)
SEQ ID NO: 32
(DP-11-01g) - c(Tyr-Gly-Gly-Phe-Asp-Arg-SSa-D(Ala)-
Arg-Pro-Lys)
SEQ ID NO: 33
(DP-11-01h) -c(Tyr-Gly-Gly-Phe-Asp-Arg-SSa-D(Leu)-
Arg-Pro-Lys)
SEQ ID NO: 34
(DP-11-01i) - c(Tyr-Gly-Gly-Phe-Asp-Arg-SSa-D(Val)-
Arg-Pro-Lys)
SEQ ID NO: 35
(DP-11-01j) - c(Tyr-Ala-Gly-Phe-Asp-Arg-SSa-D(Ala)-
Arg-Pro-Lys)
SEQ ID NO: 36
(CP1) - c(Tyr-SSa-Gly-Phe-L-Asp-Arg-Arg)
SEQ ID NO: 37
(CP2) - c(Tyr-Asp-Gly-Phe-SSa-Arg-Arg)
SEQ ID NO: 38
(CP3) -c(Tyr-Gly-SSa-Phe-Asp-Arg-Arg)
SEQ ID NO: 39
(CP4) - c(Tyr-Gly-Asp-Phe-SSa-Arg-Arg)
SEQ ID NO: 40
(CP9) -c(Tyr-Sar-(p-Cl-Phe)-Leu-Arg-D(Arg)-SSa-Asp)
SEQ ID NO: 41
(CP10) -c(Tyr-Sar-(p-Cl-Phe)-Leu-Arg-D(Arg)-SSa-
Arg-Asp-Lys)
SEQ ID NO: 42
(CP11) -c(Tyr-Sar-(p-Cl-Phe)-Leu-Arg-NMA-SSa-Arg-
Asp-Lys)
SEQ ID NO: 43
(CP12) -c(Tyr-Sar-(p-NO₂-Phe)-Leu-Arg-NMA-SSa-Arg-
Asp-Lys)
SEQ ID NO: 44
(CP13) -c(Tyr-Sar(p-NO₂-Phe)-Leu-Arg-D(Arg)-SSa-
Arg-D(Asp)-Lys
and
SEQ ID NO: 45
(CP14) -c(Tyr-Sar-(p-NO₂-Phe)-Leu-Arg-NMA-D(Asp)-
D(Arg)-SSa-D(Lys));

or a salt or stereoisomer or solvate thereof.

Compound DP-11-01c was cyclized through the L-Asp at X₅.

The compounds of the present invention may be viewed as analgesics or painkillers. The data presented in the experimental section supports this view. It is an advantage of the present compounds that they may additionally demonstrate improved metabolic stability and/or exhibit fewer or less severe side-effects when compared to dynorphin.

According to a fourth aspect, the invention resides in a pharmaceutical composition comprising a compound of any one of the first to third aspects, or a pharmaceutically acceptable salt, stereoisomer, solvate or prodrug thereof, and a pharmaceutically acceptable carrier, diluent and/or excipient.

Suitably, the pharmaceutically acceptable carrier, diluent and/or excipient may be or include one or more of diluents, solvents, pH buffers, binders, fillers, emulsifiers, disintegrants, polymers, lubricants, oils, fats, waxes, coatings, viscosity-modifying agents, glidants and the like.

The salt forms of the compounds of the invention may be especially useful due to improved solubility.

Diluents may include one or more of microcrystalline cellulose, lactose, mannitol, calcium phosphate, calcium sulfate, kaolin, dry starch, powdered sugar, and the like. Binders may include one or more of povidone, starch, stearic acid, gums, hydroxypropylmethyl cellulose and the like. Disintegrants may include one or more of starch, croscarmellose sodium, crospovidone, sodium starch glycolate and the like. Solvents may include one or more of ethanol, methanol, isopropanol, chloroform, acetone, methylethyl ketone, methylene chloride, water and the like. Lubricants may include one or more of magnesium stearate, zinc stearate, calcium stearate, stearic acid, sodium stearyl fumarate, hydrogenated vegetable oil, glyceryl behenate and the like. A glidant may be one or more of colloidal silicon dioxide, talc or cornstarch and the like. Buffers may include phosphate buffers, borate buffers and carbonate buffers, although without limitation thereto. Fillers may include one or more gels inclusive of gelatin, starch and synthetic polymer gels, although without limitation thereto. Coatings may comprise one or more of film formers, solvents, plasticizers and the like. Suitable film formers may be one or more of hydroxypropyl methyl cellulose, methyl hydroxyethyl cellulose, ethyl cellulose, hydroxypropyl cellulose, povidone, sodium carboxymethyl cellulose, polyethylene glycol, acrylates and the like. Suitable solvents may be one or more of water, ethanol, methanol, isopropanol, chloroform, acetone, methylethyl ketone, methylene chloride and the like. Plasticizers may be one or more of propylene glycol, castor oil, glycerin, polyethylene glycol, polysorbates, and the like.

Reference is made to the Handbook of Excipients 6th Edition, Eds. Rowe, Sheskey & Quinn (Pharmaceutical Press), which provides non-limiting examples of excipients which may be useful according to the invention.

It will be appreciated that the choice of pharmaceutically acceptable carriers, diluents and/or excipients will, at least in part, be dependent upon the mode of administration of the formulation. By way of example only, the composition may be in the form of a tablet, capsule, caplet, powder, an injectable liquid, a suppository, a slow release formulation, an osmotic pump formulation or any other form that is effective and safe for administration.

Suitably, the pharmaceutical composition is for the treatment of pain.

In a fifth aspect, the invention resides in a method of treating or preventing pain in a subject including the step of administering a therapeutically effective amount of a compound of any one of the first to third aspects, or a pharmaceutically acceptable salt, stereoisomer, solvate or prodrug thereof, or the pharmaceutical composition of the fourth aspect, to the subject to thereby treat or prevent pain.

In a sixth aspect, the invention resides in the use of a compound of any one of the first to third aspects, or a pharmaceutically acceptable salt, stereoisomer, solvate or prodrug thereof, or the pharmaceutical composition of the fourth aspect, in the manufacture of a medicament for the treatment or prevention of pain.

In a seventh aspect, the invention resides in a compound of any one of the first to third aspects, or a pharmaceutically acceptable salt, stereoisomer, solvate or prodrug thereof, or the pharmaceutical composition of the fourth aspect, for use in the treatment or prevention of pain.

In an eighth aspect, the invention resides in a molecule comprising a compound of any one of the first to third aspects.

As generally used herein, the terms “administering” or “administration”, and the like, describe the introduction of the compound or composition to a subject such as by a particular route or vehicle. Routes of administration may include topical, parenteral and enteral which include oral, buccal, sub-lingual, nasal, anal, gastrointestinal, subcutaneous, intramuscular, intravenous and intradermal routes of administration, although without limitation thereto.

By “treat”, “treatment” or treating” is meant administration of the compound or composition to a subject to at least ameliorate, reduce or suppress pain experienced by the subject.

By “prevent”, “preventing” or “preventative” is meant prophylactically administering the formulation to a subject who does not exhibit experience pain, but who is expected or anticipated to likely experience pain in the absence of prevention.

As used herein, “effective amount” refers to the administration of an amount of the relevant compound or composition sufficient to prevent the experience of pain, or to bring about a halt in experiencing pain or to reduce the extent of the pain experienced. The effective amount will vary in a manner which would be understood by a person of skill in the art with patient age, sex, weight etc. An appropriate dosage or dosage regime can be ascertained through routine trial.

As used herein, the terms “subject” or “individual” or “patient” may refer to any subject, particularly a vertebrate subject, and even more particularly a mammalian subject, for whom treatment is desired. Suitable vertebrate animals include, but are not restricted to, primates, avians, livestock animals (e.g., sheep, cows, horses, donkeys, pigs), laboratory test animals (e.g., rabbits, mice, rats, guinea pigs, hamsters), companion animals (e.g., cats, dogs) and captive wild animals (e.g., foxes, deer, dingoes). A preferred subject is a human.

Suitably, the pain being treated is selected from the group consisting of nociceptive pain, somatic pain, visceral pain, neuropathic pain, pain syndrome, diabetic neuropathy, trigeminal neuralgia, postherpetic neuralgia, post-stroke pain, complex regional pain syndrome, reflex sympathetic dystrophy, causalgias, cancer pain, acute pain, chronic pain, inflammatory pain and psychogenic pain. Any condition for which dynorphin is considered an appropriate treatment or co-treatment may be considered suitable for treatment using a compound of the first to third aspects or the composition of the fourth aspect.

### EXAMPLES AND EXPERIMENTAL

### In Silico Docking Studies

A number of compounds within the scope of the invention were constructed using PerkinElmer ChemBio3D version 14.0 software. Amino acids were selected from templates and their α-amino and carboxy termini linked from C-to-N terminus to form the desired peptide 2D structures were converted into energy minimised 3D structures using embedded Merck Molecular Force Field (MMFF94) software. All peptide structures were then saved in Protein Data Bank (.pdb) format.

Affinity studies were conducted using UCSF Chimera with Autodock Vina software. These compounds were programmed to dock to designated receptor sites (i.e. KOP, DOP and MOP, respectively) based on the search volume (see Table 1). Receptor structures were obtained from the RCSB PDB website. Affinity scores and hydrogen bonds for each study were performed in triplicate and recorded.

TABLE 1
In silico docking studies for compounds of the present invention (Dynorphin 1-7 analogues)

| | | Score | | | H-bonds found | | |
|---|---|---|---|---|---|---|---|
| Code | Sequence | MOP | DOP | KOP | MOP | DOP | KOP |
| Endomorphin- [symbol] | Tyr-Pro-Trp-Phe-NH₂ | −10.90 | −8.30 | −10.70 | 19 | 9 | 16 |
| DAMGO | Tyr- [symbol] (Ala)-Gly-N-MePhe-Gly-OH | −9.00 | −7.60 | −8.60 | 12 | 13 | 18 |
| 5-nitroquinoline | | −6.00 | −6.20 | −6.20 | 2 | 0 | 0 |
| CR 845 | O(Phe-phe-Leu-Lys)-4-Pip(NH₂)-OH | −8.60 | | | 15 | | 31 |
| DP-7-00 | [symbol] -(Tyr-Gly-Gly-Phe-Leu-Arg-Arg) | −9.00 | | −9.60 | 36 | | 57 |
| DP-7-06a | [symbol] (Tyr- [symbol] -Gly- [symbol] (Phe)-Asp-Arg-Arg) | 2.00 | 1.50 | −8.60 | 18 | 11 | 30 |
| DP-7-07a | [symbol] (Tyr-Asp-Gly-D(Phe)- [symbol] -Arg-Arg) | 4.70 | 4.20 | −8.00 | [symbol] 9 | 14 | 22 |
| DP-7-08a | c(Tyr-Gly- [symbol] -D(Phe)-Asp-Arg-Arg) | 1.00 | −3.10 | −9.00 | 42 | 7 | 27 |
| DP-7-09a | c(Tyr-Gly-Asp-D(Phe)-SS [symbol] -Arg-Arg) | −2.90 | 6.90 | −7.70 | 13 | 18 | 24 |
| DP-7-10 | c(Tyr-D(SSa)-Gly-Phe- [symbol] (Asp)-Arg-Arg) | | | | | | |
| DP-7-11a | c(Tyr-SSa-Gly-Phe-D(Asp)-D(Arg)-Arg) | −2.40 | −1.50 | −8.60 | 19 | 14 | 34 |
| DP-7-11b | c(Tyr-SSa-Gly-Phe-D(Asp)-Arg-D(Arg)) | −2.00 | 4.80 | −8.40 | 17 | 38 | 26 |
| DP-7-11c | c(Tyr-SSa-Gly-Phe-D(Asp)-D(Arg)-D(Arg)) | −4.40 | −2.40 | −9.30 | [symbol] 8 | 7 | 26 |
| DP-7-11 | c(Tyr-SSa-Gly-Phe-D(Asp)-Arg-Arg) | 2.20 | 5.80 | −9.90 | 47 | 23 | 10 |
| DP-7-12 | c(Tyr- [symbol] (Asp)-Gly-Phe-SS [symbol] -Arg-Arg) | 2.60 | | −8.70 | 5 | | 32 |
| DP-7-13 | c(Tyr-Gly-SSa-Phe-D(Asp)-Arg-Arg) | −2.10 | −1.70 | −8.80 | 41 | 9 | 37 |
| DP-7-14 | c(Tyr-Gly-D(Asp)-Phe-SS [symbol] -Arg-Arg) | −5.40 | −1.50 | −9.40 | 18 | 14 | 35 |

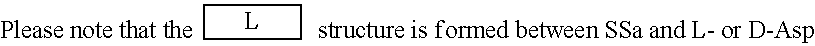

[symbol]

indicates data missing or illegible when filed

TABLE 1a
In silico docking studies for compounds of the present invention (Dynorphin 1-9 analogues)

| | | Score | | | H-bonds found | | |
|---|---|---|---|---|---|---|---|
| Code | Sequence | MOP | DOP | KOP | MOP | DOP | KOP |
| DP-9-01a | c(Tyr-Gly-Gly-Phe- [symbol] (Asp)-Arg-SSa-D(Leu)-Arg) | −2.3 | 8.5 | −8.2 | 5 | 1.5 | 35 |
| DP-9-01b | c(Tyr-Gly-Gly-Phe [symbol] (Asp)-Arg-SSa-D(Val)-Arg) | −4.7 | 7.9 | −8.75 | 5.5 | 3.5 | 3 |
| DP-9-01c | c(Tyr-Gly-Gly-Phe [symbol] (Asp)-Arg-SSa-D(Phe)-Arg) | −1.7 | 8.3 | −9.1 | 4.5 | 0.5 | 2.5 |
| DP-9-02a | c(Tyr-Gly-Gly-PheSSa-Arg-D(Asp)-D(Phe)-Arg) | 3.65 | 2.3 | −8.8 | 2 | 3 | 3 |
| DP-9-03a | c(Tyr-Gly-Gly-Phe-Leu-Arg- [symbol] 6a-D(Phe)-Asp) | −5.25 | 5.05 | −10 | 4 | 0.5 | 3.5 |
| DP-9-03b | c(Tyr-Gly-Gly-Phe-Leu-Arg- [symbol] Sa-D(val)-Asp) | −5.05 | 4.6 | −9.8 | 4.5 | 2 | 3.5 |
| DP-9-04a | c(Tyr-SSa-Gly-Phe-Asp-Arg-Arg-D(Val)-Arg) | −0.8 | 12.1 | −6.6 | 4.5 | 2 | 3.5 |

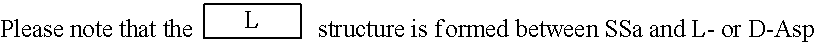

[symbol]

indicates data missing or illegible when filed

TABLE 1b
In silico docking studies for compounds of the present invention (Dynorphin 1-11 analogues)

| Code | Sequence | MOP | DOP | KOP | MOP | DOP | KOP |
|---|---|---|---|---|---|---|---|
| DP-11-01 | c(Tyr-Gly-Gly-Phe-Asp-Arg-SSa-Ile-Arg-Pro-Lys) | 0.3 | 18.1 | −8.8 | 12 | 2 | 5 |
| DP-11-02 | c(Tyr-Gly-Gly-Phe-Asp-Arg-Arg-SSa-Arg-Pro-Lys) | 5.2 | 17.1 | −3.5 | 7 | 1 | 2 |
| DP-11-03 | c(Tyr-Gly-Gly-Phe-Asp-Arg-Arg-Ile-SSa-Pro-Lys) | 9.7 | 19.1 | −4.6 | 5 | 2 | 1 |
| DP-11-04 | c(Tyr-Gly-Gly-Phe-Asp-Arg-Arg-Ile-Arg-SSa-Lys) | 2.6 | 26.8 | −9 | 9 | 5 | 4 |
| DP-11-05 | c(Tyr-Gly-Gly-Phe-Asp-Arg-Arg-Ile-Arg-Pro-Ssa) | 7.1 | 41.1 | −5.9 | 8 | 4 | 3 |
| DP-11-06 | c(Tyr-Gly-Gly-Phe-D(Asp)-Arg-SSa-Ile-Arg-Pro-Lys) | 1.7 | 15.7 | −8.3 | 4 | 2 | 7 |
| DP-11-01a | c(Tyr-Ala-Gly-Phe-Asp-Arg-SSa-Ile-Arg-Pro-Lys) | 3.2 | 21 | −8.2 | 5 | 6 | 4 |
| DP-11-01b | c(Tyr-Gly-Ala-Phe-Asp-Arg-SSa-Ile-Arg-Pro-Lys) | 2.4 | 14.5 | −7.7 | 5 | 0 | 6 |
| DP-11-01c | c(Tyr-Gly-D(Asp)-Phe-Asp-SSa-Ile-Arg-Pro-Lys | 6 | 19.3 | −6.6 | 5 | 7 | 2 |
| DP-11-01d | c(Tyr-Gly-Gly-Trp-Asp-SSa-Ile-Arg-Pro-Lys) | 2.6 | 20.9 | −7.3 | 7 | 1 | 2 |
| DP-11-01e | c(Tyr-Gly-Gly-Tyr-Asp-Arg-SSa-Ile-Arg-Pro-Lys) | 1.8 | 15.2 | −7.1 | 8 | 3 | 4 |
| DP-11-01f | c(Tyr-Gly-Gly-Phe-Asp-Arg-SSa-Ala-Arg-Pro-Lys) | 3.4 | 10.3 | −7.7 | 6 | 4 | 3 |
| DP-11-01g | c(Tyr-Gly-Gly-Phe-Asp-Arg-SSa-D(Ala)-Arg-Pro-Lys) | 0.4 | 14.5 | −8.7 | 6 | 7 | 7 |
| DP-11-01h | c(Tyr-Gly-Gly-Phe-Asp-Arg-SSa-D(Leu)-Arg-Pro-Lys) | 0.8 | 16.6 | −8 | 6 | 6 | 6 |
| DP-11-01i | c(Tyr-Gly-Gly-Phe-Asp-Arg-SSa-D(Val)-Arg-Pro-Lys) | 0.3 | 18.3 | −7.9 | 5 | 5 | 4 |
| DP-11-01j | c(Tyr-Ala-Gly-Phe-Asp-Arg-SSa-D(Ala)-Arg-Pro-Lys) | 1.1 | 12.4 | −7.4 | 4 | 4 | 3 |

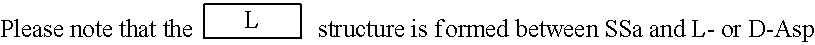

### Synthesis of Compounds of the Present Invention

### General Peptide Synthesis

Peptide synthesis was carried out on Rink amide AM resin (0.60 meq/g). All required Fmoc protected amino acids were carefully weighed into 25 mL vials and dissolved in the required quantity of dimethylformamide (DMF). Oxyma Pure (0.5 M) and diisopropylcarbodiimide (DIC; 0.5 M) were used for sequential coupling of amino acids. All coupling reactions were performed under microwave conditions except for Asp, SSa and Arg residues which were coupled at room temperature. Fmoc deprotection was performed using 20% v/v piperidine in DMF. To prevent the aspartamide formation in the case of Asp, 1% formic acid in 20% v/v piperidine was used for Fmoc deprotection. Separately, for on-resin cyclization reactions, orthogonally protected —ODmab and -Dde groups were removed using hydroxylamine hydrochloride and imidazole (1.3:1 milliequivalence in NMP). After completion of synthesis, the dry resin was collected from the synthesizer and the peptide was cleaved off-resin using the cleavage cocktail (TFA:TIPS:H₂O:DCM, 90:2.5:2.5:5). Crude peptide was collected and further purified by a preparative HPLC system using an Agilent 1200 Chem Station equipped with binary pumps and auto-fraction collector. A Jupiter 10 μm Proteo 90 Å LC column 250×21.2 mm was used with a flow rate of 10 mL/min. The mobile phase employed was MilliQ water and acetonitrile, both containing 0.1% v/v TFA with a gradient flow of 0% to 100% acetonitrile in 60 min.

### Solid Phase Synthesis of DP-7-11 and DP-7-12

An automated Biotage Peptide Synthesizer was used to synthesize DP-7-11 and DP-7-12. Standard Fmoc-chemistry was used for the synthesis of peptides, where 0.5 M HBTU in DMF and DIPEA were used as the coupling reagents, and 20% v/v piperidine in DMF as the Fmoc-deprotecting agent.

Synthesis of DP-7-11 and DP-7-12 was performed by automated synthesis, followed by cyclization performed manually. Manual deprotection of two side-chain protecting groups was performed using 1% v/v TFA in DCM, which prepared the resin-bound peptide for site-selective cyclization using standard coupling reagents. Fmoc deprotection of any base labile semi-permanent protecting groups was performed prior to thoroughly washing the resin with DMF, then DCM (2-3 resin volumes) and drying in vacuo. The dried resin was transferred to a 50 mL round-bottomed flask and cleavage reagent mixture added (TFA/DCM/TIPS/H₂O/DCM—90:5:2.5:2.5; 10 mL), with vigorous stirring for 3-4 hours at room temperature. The resin mixture was then vacuum filtered and the filtrate evaporated in vacuo, followed by azeotroping with toluene (3×15 mL) to remove residual TFA. The resulting sticky (off-white) residue was triturated with ice cold diethyl ether (5×10 mL) and then dissolved in water and lyophilised, in preparation for HPLC/MS analysis and HPLC purification.

### Generic Protocol for Synthesis of DP-7-11 and DP-7-12

The general protocol for synthesis of DP-7-11 is set out below:

1. Rink amide resin (0.100 g, loading capacity 0.34 mmol/g)

4. Fmoc-Arg (Pbf)-OH (0.066 g)

7. Repeated amino acid coupling with

a. Fmoc-Arg(Pbf)-OH (0.066 g)

b. Fmoc-SSa(Mtt)-OH (0.071 g)

c. Fmoc-Phe-OH (0.048 g)

d. Fmoc-Gly-OH (0.048 g)

e. Fmoc-Asp(PhiPr)—OH (0.048 g)

f. Fmoc-Tyr-OH (0.041 g, 3 eqw.r.t original resin loading)

The cyclization reaction was performed after 7e, a separate deprotection reaction was used with 3% TFA (DCM) for 5 min and then the cyclization reaction was performed between side chain groups. To synthesis DP-7-12, 7b and 7e amino acids were added alternatively.

### Example Synthesis of DP-7-11 and DP-7-12

DP-7-11 and DP-7-12 were also prepared wholly on-resin, using well-established Fmoc-SPPS (see Table 2). Each construct was prepared by replacing the 2^{nd} and 5^{th} amino acids of the sequence with Asp or SSa, with the general structure: NH₂-Tyr-c(Xaa-Gly-Phe-Yaa)-Arg-Arg-CONH₂ (Xaa=Asp or SSa, Yaa=Asp or SSa). Cyclization was carried out between the side-chain amino group of SSa and the carboxylic group of Asp, which were first deprotected of Mtt and PhiPr, respectively, under mildly acidic conditions, prior to cyclisation using standard activation reagents. The last residue Tyr was then coupled to the cyclised peptide prior to cleavage off-resin, purification and characterisation, which confirmed the presence of the target DP-7-11 in good yield (≈55%).

The synthesized DP-7-11 and DP7-12 were compared to dynorphin 1-7 (herein referred to also as “DP-7-00”). Furthermore, DP-11-00 and DP-11-06 were synthesized and DP-11-06 was compared to dynorphin 1-11 (herein referred to as ‘DP-11-00’). DP-7-00 and DP-11-00 could be synthesized using solid phase peptide synthesis.

TABLE 2
DP-7-00, DP-7-11, DP-7-12, DP-11-00, DP-11-06

| Serial No | Descriptor | Sequence |
|---|---|---|
| DP-7-00 | Dyn 1-7 | Tyr-Gly-Gly-Phe-Leu-Arg-Arg |
| DP-7-11 | Dyn 1-7; 2-SSa, 5-D(Asp) | c(Tyr-SSa-Gly-Phe-D(Asp)-Arg-Arg) |
| DP-7-12 | Dyn 1-7; 2-D(Asp), 5-SSa | c(Tyr- D(Asp)--Gly-Phe-SSa-Arg-Arg) |
| DP-11-00 | Dyn 1-11 | Tyr-Gly-Gly-Phe-Leu-Arg-Arg-Ile-Arg-Pro-Lys |
| DP-11-06 | Dyn 1-11; 5-D(Asp), 7-SSa | c(Tyr-Gly-Gly-Phe-D(Asp)-Arg-SSa-Ile-Arg-Pro-Lys) |

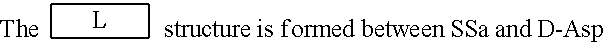

### Analysis and Purification of DP-7-00, DP-7-11, DP-7-12, DP-11-00 and DP-11-06

The relative purity of the crude/purified peptide was assessed using a Shimadzu Nexera-i LC-2040C 3D liquid chromatography instrument equipped with a C₁₈ column (Vydac 214TP, 5μ, and length 250×4.6 mm ID) and using a solvent gradient (solvent A: 0.1% v/v TFA_{(aq)}; solvent B: 0.1% v/v TFA in ACN—see Table 3 for gradient conditions) with flow rate of 1 mL/min and monitored at 219 nm. A blank run (solvent only) was conducted between each sample.

TABLE 3
Gradient for analytical HPLC.

| Time (minutes) | Solvent A (%) | Solvent B (%) |
|---|---|---|
| 0-3 | 100 | 0 |
| 3-30 | 50 | 50 |
| 30-35 | 0 | 100 |
| 35-40 | 0 | 100 |
| 40-55 | 100 | 0 |

Preparative HPLC: An Agilent Chem Station consisting of an Agilent Binary HPLC preparative pump and fraction collector was used to purify crude peptides. Separation of target peptides was performed on a Jupiter Proteo 90 Å LC column (10 μm, 250×21.2 mm) using a solvent gradient (solvent A: 0.1% v/v TFA_{(aq)}; solvent B: 0.1% v/v TFA in ACN—see Table 4 for gradient conditions). Prior to purification the column was equilibrated with an initial mobile phase condition of 90:10 (solvent A: solvent B) for 15 minutes.

TABLE 4
Gradient for preparative HPLC

| Time (minutes) | Solvent A (%) | Solvent B (%) |
|---|---|---|
| 0-8 | 74 | 26 |
| 8-14 | 70 | 30 |
| 14-20 | 90 | 10 |

Desired fractions from preparative HPLC were collected and confirmed for the target molecular ion using mass spectrometry (ESI-MS).

ESI-MS: Samples were analyzed using an Applied Biosystem/MDS Sciex Q-TRAP LC/MS/MS system. Sample preparation involved dissolving the peptide in 50:50 acetonitrile-water to a final concentration of ≈1 μg/mL. Declustering potential and entrancing potential were set at 200 and 10 mV, respectively. The sample infusion rate was adjusted to 10 μL/min with Q1 scan mode selected for detection of the target molecular ion. The summary of HPLC and MS data for DP-7-00, DP-7-11, DP-7-12, DP-11-00 and DP-11-06 are shown in Table 5 below.

TABLE 5
HPLC and MS details of select compounds

| | Retention | | Calculated | |
|---|---|---|---|---|
| | time | Purity | monoisotopic | |
| Compound | (min) | (%) | mass [M]⁺ | Observed [M + H]⁺ |
| DP-7-00 | 18.2 | 99.2 | 866.4875 | 434.2522 [M + 2H]²⁺ |
| DP-7-12 | 10.6 | 100.0 | 999.4531 | 1000.4568 |
| DP-7-11 | 10.8 | 100.0 | 999.4531 | 1000.4704 |
| DP-11-00 | 19.9 | 98.4 | 1360.8 | 1361.8 |
| DP-11-06 | 20.8 | 88.2 | 1394.7 | 1395.6 |

The compounds listed in Table 6 were synthesized in a similar fashion.

TABLE 6
Cyclic analogues of DynA-1-7, 1-9 and 1-11

| Codes | X1 | X2 | X3 | X4 | X5 | X6 | X7 | X8 | X9 | X10 | X11 |
|---|---|---|---|---|---|---|---|---|---|---|---|
| CP1 | Tyr | c-SSa | Gly | Phe | c-L-Asp | Arg | Arg | — | — | — | — |
| CP2 | Tyr | c-L-Asp | Gly | Phe | c-SSa | Arg | Arg | — | — | — | — |
| CP3 | Tyr | Gly | c-SSa | Phe | c-L-Asp | Arg | Arg | — | — | — | — |
| CP4 | Tyr | Gly | c-L-Asp | Phe | c-SSa | Arg | Arg | — | — | — | — |
| CP5 or DP-7-11 | Tyr | c-SSa | Gly | Phe | c-D-Asp | Arg | Arg | — | — | — | — |
| CP6 or DP-7-12 | Tyr | c-D-Asp | Gly | Phe | c-SSa | Arg | Arg | — | — | — | — |
| CP7 or DP-7-13 | Tyr | Gly | c-SSa | Phe | c-D-Asp | Arg | Arg | — | — | — | — |
| CP8 or DP-7-14 | Tyr | Gly | c-D-Asp | Phe | c-SSa | Arg | Arg | — | — | — | — |
| CP9 | Tyr | — | Sar | p-Cl-Phe | Leu | Arg | D-Arg | c-SSa | c-L-Asp | — | — |
| CP10 | Tyr | — | Sar | p-Cl-Phe | Leu | Arg | D-Arg | c-SSa | Arg | c-L-Asp | Lys |
| CP11 | Tyr | — | Sar | p-Cl-Phe | Leu | Arg | NMA | c-SSa | Arg | c-L-Asp | Lys |
| CP12 | Tyr | — | Sar | p-NO₂-Phe | Leu | Arg | NMA | c-SSa | Arg | c-L-Asp | Lys |
| CP13 | Tyr | — | Sar | p-NO₂-Phe | Leu | Arg | D-Arg | c-SSa | Arg | c-D-Asp | Lys |
| CP14 | Tyr | — | Sar | p-NO₂-Phe | Leu | Arg | NMA | c-D-Asp | D-Arg | c-SSa | D-Lys |

Tyr—tyrosine; SSa—disulfide linker amino acid, as illustrated above; Gly—glycine; Phe—phenylalanine; D-Phe—D-phenylalanine; Asp—aspartic acid; Arg—arginine; D-Arg—D-arginine; Sar—sarcosine; Ile—isoleucine; D-Val—D-valine; p-Cl-Phe—p-chlorophenylalanine; p-NO₂-Phe—p-nitrophenylalanine; NMA—N(α)-methylarginine; Lys—lysine; D-Lys—D-lysine; c—point of cyclisation (i.e. amino group of SSa forms an amide bond with the side-chain carboxylic acid of Asp). All the amino acids are L-isomer unless stated otherwise.

The compounds listed in Table 6 were purified as outlined above and analyzed by mass spectrometry. Details of the purifications and mass spectrometry is provided in Table 7.

TABLE 7
HPLC and MS data for CP1-CP14

| | HPLC purity | | | | | |
|---|---|---|---|---|---|---|
| | Retention | % | LCMS data | | | |
| Code | time(mins) | Purity | m + 1/z | m + 2/z | m + 3/z | m + TFA |
| CP1 | 16.17 | 98.55 | 1000.40 | 500.90 | 334.40 | 1114.35 |
| CP2 | 18.95 | 94.71 | 1000.30 | 501.00 | n.f | 1114.30 |
| CP3 | 16.02 | 90.65 | 1000.30 | 501.00 | n.f | 1114.30 |
| CP4 | 17.04 | 92.67 | 1000.35 | 500.95 | n.f | 1114.40 |
| CP5 | 17.75 | 90.74 | 1000.40 | 500.73 | 334.16 | 1114.45 |
| CP6 | 20.34 | 99.82 | n.f | 500.73 | 334.16 | 1114.45 |
| CP7 | 15.61 | 88.51 | 1000.56 | 500.78 | 334.19 | 1114.56 |
| CP8 | 17.46 | 91.10 | n.f | 500.73 | 334.16 | 1114.44 |
| CP9 | 21.69 | 99.50 | n.f | 581.26 | n.f | 1275.50 |
| CP10 | 19.24 | 97.53 | n.f | 723.36 | 482.57 | n.f |
| CP11 | 8.02 | 93.77 | 1459.50 | 730.90 | 487.70 | n.f |
| CP12 | 5.88* | 96.77 | 1470.60 | 736.20 | 491.20 | n.f |
| CP13 | 26.93* | 97.95 | n.f | 728.89 | 426.26 | n.f |
| CP14 | 26.71 | 99.25 | n.f | 735.91 | 490.93 | n.f |

Analytical RP-HPLC was performed on a Shimadzu Nexera-i LC-2040C 3D with a C₁₈ column (Grace Vydac 214TP, 5 μm, length 250 × 4.6 mm ID or
*Phenomenex Kinetex, 5 μm 150 × 4.6 mm ID)with a flow rate of 1 mL/min. The mobile phase employed was solvent A: MilliQ water, Solvent B: acetonitrile, both containing 0.1% v/v TFA with the gradient from 0-100% B for 45 min.
n.f. not found

### Metabolic Stability Studies

Purified DP-7-11 (1 mg/mL) was dissolved in 0.1 M ammonium bicarbonate (NH₄HCO₃) buffer. To prepare a stock trypsin solution, 1 mg trypsin was dissolved in 50 mL of 0.1 M NH₄HCO₃ buffer. Equal volumes of the stock trypsin solution (62.5 μL) and DP-7-11 solution (62.5 μL) were incubated in 375 μL of 0.1M NH₄HCO₃ buffer in a 37° C. water bath. Aliquots of 100 μL were collected from this mixture at set time intervals of 0 min, up to 24 hours. Ice-cold acetonitrile containing 0.5% TFA was used to quench the reaction between DP-7-11 and trypsin at predetermined intervals, and just prior to HPLC or LC-MS analysis. The quenched samples were vortexed for 10 minutes followed by centrifugation at 12,000 rpm for a further 15 minutes. Supernatant was sampled and analysed using analytical RP-HPLC or LC-MS. Samples without trypsin acted as negative controls and were sampled at two intervals of 0 hour and 6 hours.

Serum stability of DP-7-11 was also performed. In this regard, rat serum replaced trypsin and NH₄HCO₃ buffer. Water was used as negative control in place of serum, with the stability study performed in an identical fashion to the trypsin study. DP-7-11 was compared to DP-7-00 to determine its relative stability. DP-11-00 and DP-11-06 were tested in a similar manner.

### Serum and Trypsin Stability

As used in this serum and trypsin stability discussion, the term ‘degraded completely’ relates to the relevant compound being completely absent when tested. In other words, the compound being tested is not observed when tested. For instance, in the serum stability of DP-7-00, no DP-7-00 was observed after being incubated in serum for 1 hr.

### Serum Stability

DP-7-00 was incubated in serum at 37° C. for 24 h and samples were collected in each time point. Analysis using LC-MS showed that DP-7-00 degraded completely within 1 h. Analysis of the results of DP-7-00 suggest that complete degradation occurred within 15 minutes. Under the same conditions, DP-7-11 displayed a half-life of 6 h. This appears to indicate the improved metabolic stability of the present invention. Serum stability for DP-7-11 is shown in FIG. 2.

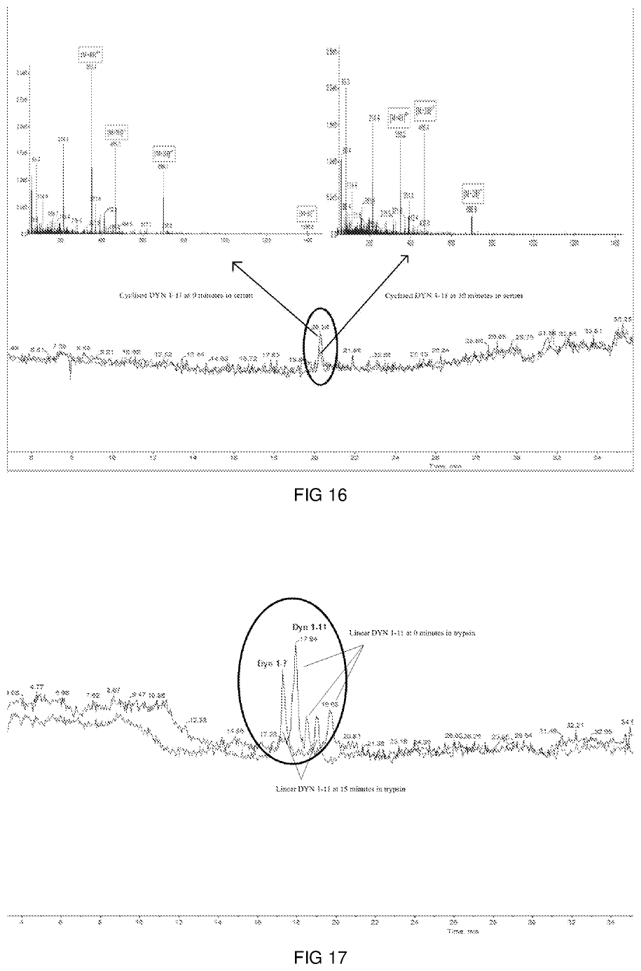

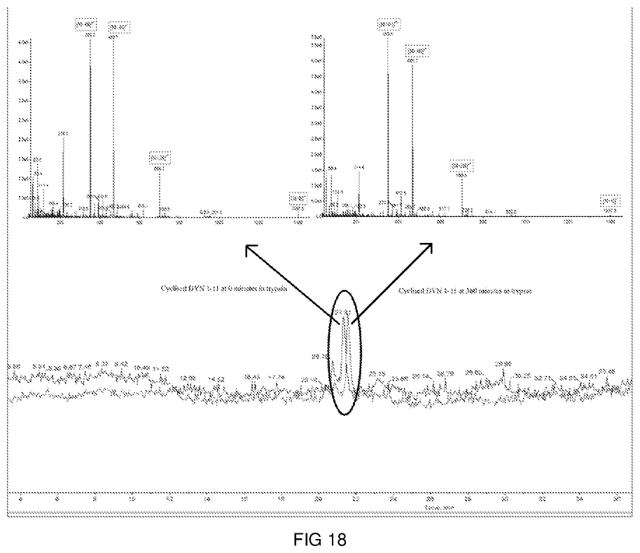

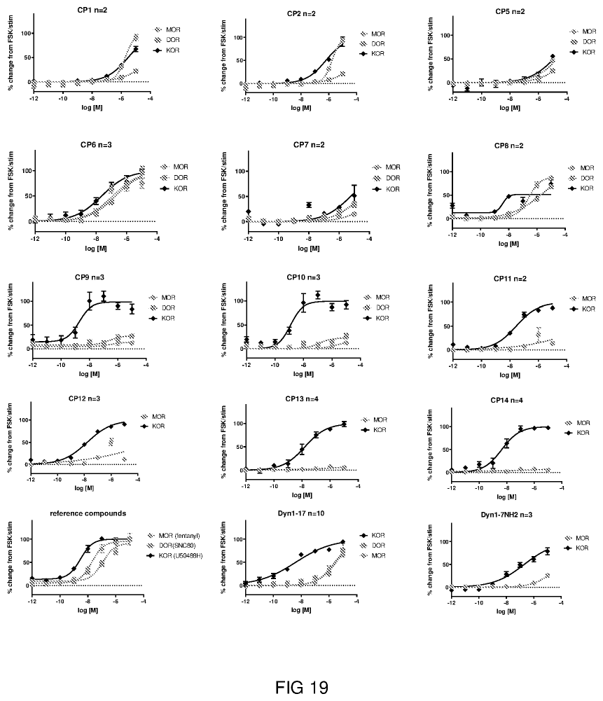

DP-11-00 was incubated in serum at 37° C. for 24 h and samples were collected in each time point. Analysis using LC-MS showed that DP-11-00 degraded completely within 1 h. Analysis of the results of DP-11-00 suggest that complete degradation may occur within 15 minutes. Under the same conditions, DP-11-06 displayed a half-life of 30 minutes. This appears to indicate the improved metabolic stability of the present invention. Serum stability for DP-11-06 is shown in FIG. 16.

### Trypsin Stability

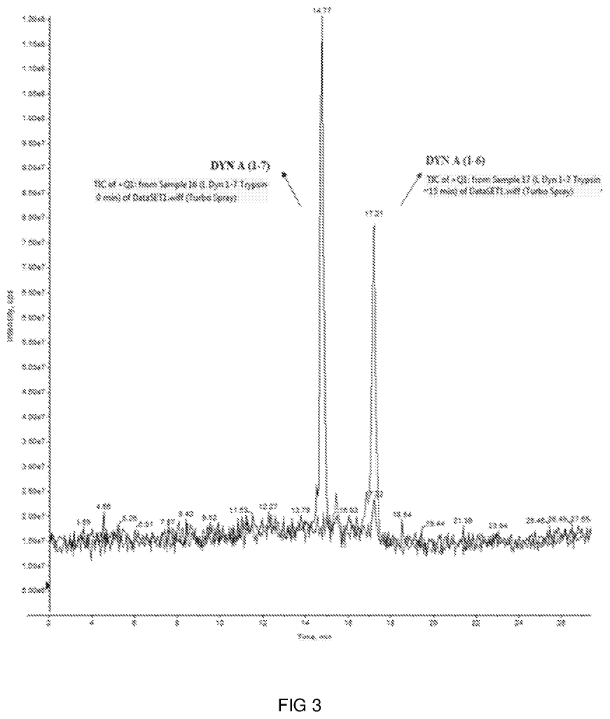

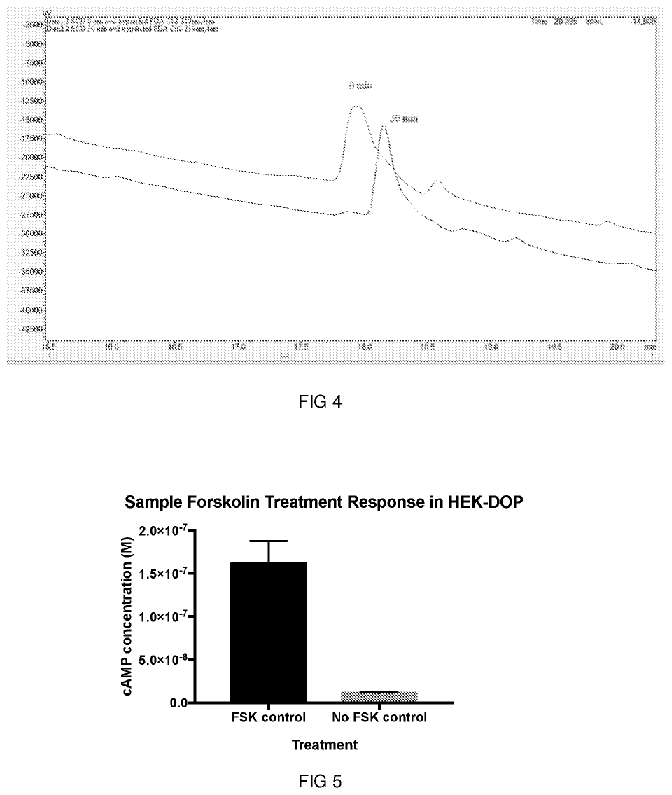

DP-7-00 and DP-11-00 were highly susceptible to trypsin digestion. The retention time of DP-7-00 was found to be 14.77 min. After 15 min of incubation with trypsin, no peak corresponding to DP-7-00 was observed and a new peak with a retention time of 17.21 min appeared. The fragmentation pattern suggested that this new peak corresponds to the less polar compound DYN A (1-6). This kind of fragmentation was not observed in negative control sample indicating that the conversion was solely due to trypsin. A similar cleavage pattern was observed in case of DP-11-00, resulting in DYN A (1-7) and DYN A (1-6) in initial time point samples. It is postulated that this is due to the fact that trypsin specifically cleaves at the C-terminal of arginine and lysine residues unless followed by proline as in case of DP-11-00 where DYN 1-9 was not observed upon trypsin digestion. The trypsin stability for DP-7-00 is shown in FIG. 3. Under the same conditions, DP-7-11 also experiences complete degradation within 1 hr. This appears to be consistent with DP-7-00. Trypsin stability for DP-7-11 is shown in FIG. 4.

In DP-11-06, when a disulfide bridge was placed next, i.e., C-terminus to arginine as in case, the vast majority of intact peptide was observed over a period of at least 6 h. This appears to indicate the improved metabolic stability of the present invention.

The results of serum and trypsin stability are shown in Table 8 below.

TABLE 8
Serum and trypsin stability comparisons of DP-7-11
and DP-7-00, and DP-11-06 and DP-11-00

| Dyn analogs | Serum | Trypsin |
|---|---|---|
| DP-7-00 | Complete degradation | Complete degradation |
| | within 1 h | within 1 h |
| DP-7-11 | Half-life 6 h | Complete degradation |
| | | in 1 h |
| DP-11-00 | Complete degradation | Complete degradation |
| | within 1 h | within 1 h |
| DP-11-06 | Half-life 30 min | Stable up to 6 h |

### Serum Stability of DP-7-12

The serum stability of DP-7-12 was completed. The results suggest that approximately 67% of DP-7-12 was still present after 1 hr, and approximately 20% of the DP-7-12 was still present after 2 hours. This appears to indicate the improved metabolic stability of the present invention.

### Serum and Trypsin Stability Monitored Using LC-MS

The serum and trypsin stability of DP-7-11 and DP-7-12 were tested (in some instances again) using LC-MS. The results of this testing are found in Table 8a. The LC-MS utilized in this study was more sensitive than the LC-MS utilized in the above tests. These results were compared to the previous DP-7-00 results.

TABLE 8a
Serum and trypsin stability comparisons of
DP-7-11 and DP-7-12 with DP-7-00

| Dyn | | Incubation in |
|---|---|---|
| Analogue | Incubation in serum | Trypsin |
| DP-7-00 | Complete degradation | Complete degradation |
| | within 15 min | within 15 min |
| DP-7-11 | Half life greater than 6 h | 25% remaining at |
| | | 30 min |
| DP-7-12 | Half-life 45 min | Complete degradation |
| | | within 30 min |

DP-7-11 was observed to be relatively stable in serum for up to 6 h, with approximately 10% degradation up to this time point. Furthermore, DP-7-11 in trypsin has approximately 25% of the peptide remaining at about 30 min. These findings indicate that DP-7-11 has improved metabolic stability when compared to uncyclized DP-7-00, which is fully degraded within this time frame.

The above results for DP-7-12 also indicate that it has improved metabolic stability when compared to uncyclized DP-7-00. In this regard, DP-7-12 showed a half life of about 45 minutes in serum whereas DP-7-00 showed complete degradation within about 15 mins (i.e. the minimum time taken to extract a sample and prepare for LC-MS evaluation).

These results appear to suggest that the relative positioning of the linker within the dynorphin sequence (and its span covering key susceptible amino acids) play a critical role in their resilience. As such, it is postulated that the position of cyclization (i.e. where the linker is formed) may play a role in the metabolic stability subsequently exhibited, particularly stability in serum and trypsin.

### Inhibition of Opioid Receptors (μ, δ, κ and Nociceptin

Opioids act via the opioid receptors (OR) which are known to predominantly couple to Gi proteins to modulate other downstream messenger molecules. In particular, opioids act as agonists at ORs, and stimulate the dissociation of the Gα and Gβy subunits in the Gi-protein. In turn, many intracellular effector pathways are propagated, including the inhibition of the enzyme adenylyl cyclase to reduce a key second messenger molecule—cyclic adenosine monophosphate (cAMP). To date, MOP remains the target of most clinically used opioids, such as morphine. Drug discovery has focused largely on MOP, as the agonism of KOP and DOP receptors have been associated with other adverse side effects.

DP-7-11 and DP-7-12 were assessed for the ability to inhibit cAMP production in HEK-DOP and HEK KOP cells.

### Materials

The HEK-DOP and HEK-KOP cell lines were provided by the University of Queensland. Forskolin 5 mg was sourced from Enzo Life Sciences® (10 Executive Blvd, Farmingdale, N.Y. 11735, United States). All cell culture and other essential materials were sourced through Sigma-Aldrich© (Castle Hill New South Wales, Australia). The AlphaScreen® cAMP kit was obtained from PerkinElmer© (Melbourne, Victoria, Australia)

### HEK-Cell Culture

The HEK-293 DOP (HEK-DOP) and HEK-293 KOP (HEK-KOP) cell lines were cultured in a T75 flask, in Dulbeco's Modified Eagle's Medium (DMEM) complete with 10% (v/v) fetal bovine serum (FBS) and 1% (v/v) Geneticin. Cells were incubated in a humidified atmosphere of 37° C. (95% air and 5% CO₂). Cells were passaged at 80-90% confluence and media was changed every two days.

### Preparation Method and Procedure

Preparation of Buffers for cAMP Assay

Stimulation buffer and Lysis buffer were prepared fresh on the day of each assay. Stimulation buffer contained 19.5 mL Hanks buffered saline solution, Bovine serum albumin (BSA) 0.1% (w/v), 0.5 mM 3-Isobutyl-1-methylxanthine (IBMX) and 5 mM HEPES (4-(2-hydroxyethyl)-1-piperazineethanesulfonic acid). Lysis buffer contained 19 mL Mili-Q H₂O, BSA 0.1% (w/v), 0.3% (v/v) 10% Tween-20, and 5 mM HEPES. Both buffers were adjusted to pH 7.4 with NaOH.

Preparation of Standard cAMP Curve

The cAMP standard dilution series was prepared from the 50 μM cAMP standard solution provided by the cAMP assay kit. The standard solution was vortexed before being serially diluted to provide a concentration range of 5×10⁻⁶ M to 5×10⁻¹¹ M in ½ Log intervals.

### Preparation of Forskolin

For this assay, Forskolin was optimised at 50 μM/well. 25 mM stocks were used to prepare 0.2 mM Forskolin. The concentration of Forskolin prepared was 4 times the required concentration in the well to account for further dilution in the well. 0.1 mM Forskolin solution was then made from this and used to dilute the peptide solutions.

### Preparation of Peptide Dilutions

DP-7-11 and DP-7-12 provided in powder form and reconstituted to 10 mM stock solutions and diluted to 1 mM working stocks using Mili-Q H₂O. Each peptide solution was serially diluted to give concentrations of 1×10⁻⁶ to 3×10⁻⁷ M with stimulation buffer.

### Preparation of Control Solutions

Stimulation buffer was added to the 0.2 mM Forskolin solution in a 1:1 ratio to prepare the Forskolin only treatment solution. This was the positive control. Stimulation buffer was used as the negative control.

### Cell Harvest and Cell Count

Cells were harvested from two T75 flasks on the day of experimentation. Identical protocol was used for both DOP and KOP cells. The cells were first removed from the incubator and washed with Versene®. The cells were then incubated in 2 mL of Versene® at 37° C. for 5 minutes. Following this, the mixture was made up to 5 mL with Versene® in a centrifuge tube. This was centrifuged at 1300 rpm for 2 minutes at 23° C. The supernatant was then decanted and the cells resuspended in 100 μL stimulation buffer for counting. A hemocytometer was used to count the cells. This assay required the concentration of cells to be 13300 cells/μL

Preparation of Separate Acceptor-Bead and Donor-Bead Mixtures for cAMP Assay

This method was used to conduct all cAMP Alphascreen assays. The acceptor bead mixture consisted of acceptor beads and stimulation buffer mixed according to the ratio 1:35 given in the kit. From this, the acceptor beads mixture was used to prepare separate mixtures for the cAMP standard curve and for the treatment wells of the assay. For the treatment wells, the beads were mixed with cells in a 1:1 ratio. For the cAMP standard curve, the beads were mixed with more stimulation buffer in a 1:1 ratio. The donor bead mixture consisted of donor beads, biotinylated cAMP and lysis buffer mixed in the ratio 1:3:300.

### Experimental Design

The DP series compounds were assayed as follows: The assay was performed using a 96 well ½ area plate. The different cAMP standard solutions (3 μL/well) were plated in duplicate. The different concentrations of drug (3 μL/well), and control solutions (3 μL/well) were plated in triplicate. Following this, the acceptor bead mixture (3 μL/well) was added to the respective sets—either cAMP standard curve or the treatment. This was covered and incubated on the orbital shaker for 30 minutes at room temperature. Then, the donor bead mixture (10 μL/well) was added to each well. This was incubated at room temperature overnight on the orbital shaker.

The CP series compounds were assayed as follows: The assay was performed using a 96 well ½ area plate. The different cAMP standard solutions (3 μL/well) were plated in duplicate. The different concentrations of drug (3 μL/well), and control solutions (3 μL/well) were plated in triplicate. Following this, the acceptor bead mixture (3 μL/well) was added to the respective sets—either cAMP standard curve or the treatment. This was covered and incubated on the orbital shaker for 60 minutes at 37° C. Then, the donor bead mixture (10 μL/well) was added to each well. This was incubated at room temperature overnight on the orbital shaker.

For DP-11-06 the assay was carried out in the same way as for the CP compounds except it was incubated for 30 mins at 37° C.

### Data Collection and Analysis

For the DP series compounds (except DP-11-06), the Ensight® Multimode Plate Reader was used to quantify the fluorescence units of each plate. Before reading, each plate was centrifuged at 280 g for 30 seconds. Using GraphPad Prism7® Software, cAMP concentrations were determined by fit spline/LOWESS analysis. The cAMP standard curve was used for interpolation at this point. Subsequently, the data for each trial was normalized to the highest in-trial cAMP concentration recorded using Microsoft Excel®. The data was then combined in GraphPad Prism7® to generate concentration-response curves and IC50s by non-linear regression analysis. The IC50 and IC80 for each compound was then calculated using the ‘EC anything’ protocol in GraphPad Prism7®.

For the CP series compounds and DP-11-06, the Ensight® Multimode Plate Reader was used to quantify the fluorescence units of each plate. Before reading, each plate was centrifuged at 280 g for 30 seconds. Using GraphPad Prism7® Software, cAMP concentrations were determined by fit spline/LOWESS analysis. The cAMP standard curve was used for interpolation at this point. The cAMP concentrations were then normalized to the highest cAMP concentration recorded and analyzed by one-way ANOVA for multiple comparisons. The IC50 for each compound was then calculated using GraphPad Prism7® with non-linear regression analysis using four parameter curve fitting.

### Evaluation of Opioid Receptor Mediated Pathway by Adding Naloxone

The preparation of buffers, cAMP standard curve, bead solutions were carried out identically to the agonist assay.

Preparation of Forskolin for cAMP Assay with Naloxone

Forskolin had been optimised at 50 μM/well. Thus, the 25 mM stocks were used to prepare 300 μL of 0.3 mM Forskolin. The concentration of Forskolin prepared was 6 times the required concentration in the well to account for further dilution in the well. Forskolin solution was then made from this and used to dilute the drug solutions.

Preparation of Peptide Dilutions for cAMP Assay with Naloxone

Approximate IC80 values were used for DP-7-11 and DP-7-12 to determine the ability of naloxone to reverse agonist inhibitory effect.

### Preparation of Naloxone

The desired concentration of naloxone was 100 μM/well. Thus, 100 μL of 600 μM naloxone was made up from 100 mM stock. This was 6 times the desired in-well concentration to account for further dilution in the well by Forskolin, peptide, cells and acceptor bead solutions.

### Preparation of Control Solutions

Stimulation buffer was added to the 0.3 mM Forskolin solution to prepare the Forskolin only treatment solution as the positive control. Stimulation buffer used as the negative control.

### Experimental Design

The DP series compounds were assayed as follows: The different cAMP standard solutions (3 μL/well) were plated in duplicate. Naloxone solution was then plated (1 μL/well) for each treatment (DP-7-11, DP-7-12, Forskolin only and stimulation buffer) in triplicate. In the same way, stimulation buffer was plated (1 μL/well) for the same number of wells. This made up two sets of wells, antagonist and non-antagonist. Following this, the acceptor bead and cell mixture (3 μL/well) was added to the treatment wells. The plates were then covered and centrifuged at 280 g for 30 seconds before incubation on an orbital shaker for 30 minutes at room temperature. The acceptor bead mixture (3 μL/well) was then added to the cAMP standard curve wells, whilst the drug mixed with Forskolin solutions (3 μL/well) were added to the respective treatment wells in triplicate. Again, the plates were centrifuged at 280 g for 30 seconds, then covered and incubated on the orbital shaker for another 30 minutes. Finally, the donor bead mixture (10 μL/well) was added to each well. This was incubated at room temperature overnight on the orbital shaker.

### Data Collection and Analysis

The Ensight® Multimode Plate Reader was used to quantify the fluorescence units of each plate. Before reading, each plate was centrifuged at 280 g for 30 seconds. Using GraphPad Prism7® Software, cAMP concentrations were determined from the fluorescence data by fit spline/LOWESS analysis. The cAMP standard curve was used for interpolation at this point. The cAMP concentrations were then normalized to the highest cAMP concentration recorded and analyzed by one-way ANOVA for multiple comparisons. This analysis was corrected for multiple comparisons using Bonferroni. This produced p-values reflecting the significance of the difference between each antagonist group and non-antagonist group.

### Results

### Establishing the Baseline for Interpreting Data Collected in Assays

It is known that DOP and KOP receptors are G protein coupled receptors which, when activated by agonists, stimulate a decrease in cAMP production via the Gi/o protein and subsequently Adenylyl cyclase modulation, amongst other effector pathways. Nevertheless, the modulation of cAMP has become a key pathway studied in the development of opioids with lowered adverse effects. The current model of efficacy screening uses the ability of experimental compounds to inhibit the Forskolin-induced cAMP production of cells as the response variable in quantitating the efficacy of such compounds as potential analgesics. Forskolin is used to induce cAMP production because of its known ability to specifically stimulate adenylyl cyclase, and hence cAMP production.

In order to test for equivalent DOP and KOP efficacy by DP-7-11 and DP-7-12, the study used HEK293 cells transfected with either DOP or KOP to assess and compare each compound's inhibitory activity on Forskolin-induced cAMP levels.

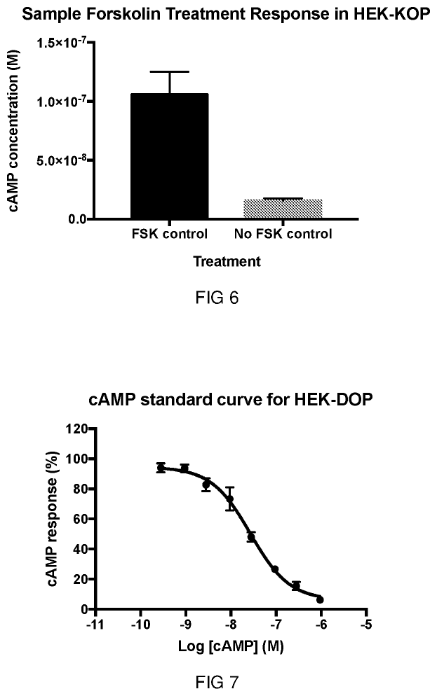

As positive and negative controls for the experiment, Forskolin and no-Forskolin treatment response was measured in each assay respectively (see FIG. 5 for DOP and FIG. 6 for KOP). Both FIGS. 5 and 6 show that the Forskolin treatment achieved a much higher concentration of cAMP, measuring about 10-fold of the no-Forskolin treatment in both cell lines. The average response in DOP was 1.61494×10⁻⁷ M and 1.13386×10⁻⁸M for Forskolin and no Forskolin respectively (FIG. 5), whilst KOP had an average response of 1.0564×10⁻⁷M and 1.57871×10⁻⁸M for Forskolin and no Forskolin respectively (FIG. 6). Unpaired t-tests confirmed these differences to be statistically significant for both DOP (FIG. 5, p<0.0001) and KOP (FIG. 6, p=0.0003).

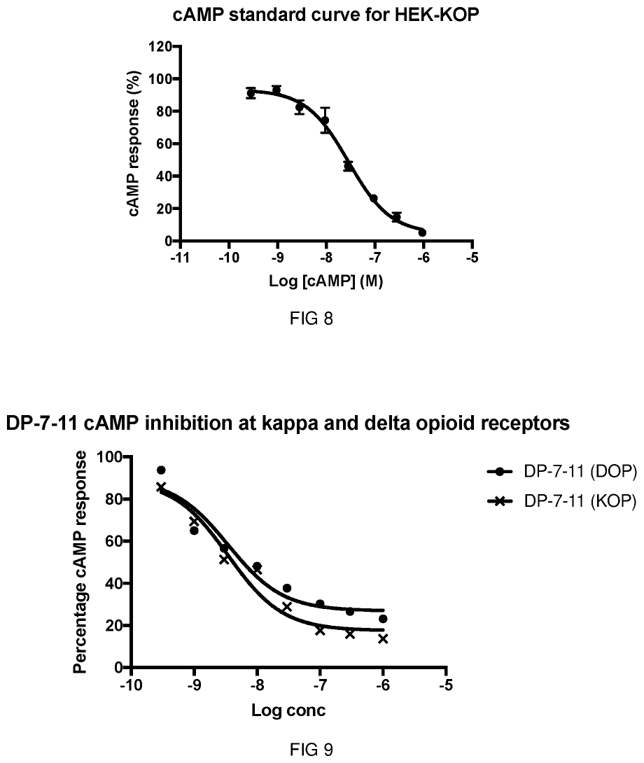

cAMP standard curves were performed with each assay. FIGS. 7 and 8 represent the average data of all standard curves produced for DOP and KOP respectively. Both standard curves show maximum cAMP response at the lowest concentration of exogenous cAMP (FIGS. 7 and 8). This response is shown to decrease in an inverse sigmoidal trend until finally the minimum cAMP response is reached at the highest concentration of exogenous cAMP (FIGS. 7 and 8). The IC50 (±95% Cl) for the standard curves produced a value of 2.701×10⁻⁸ M (1.849×10⁻⁸ to 3.96×10⁻⁸ M) for DOP (FIG. 7) and 2.850×10⁻⁸ M (1.988×10⁻⁸ to 4.107×10⁻⁸M) for KOP (FIG. 8). This value is consistent with data expected by PerkinElmer©(20) and confirms the ability of the AlphaScreen® cAMP assay to quantify levels of cAMP.

Agonist Effects on Forskolin Induced Production of cAMP

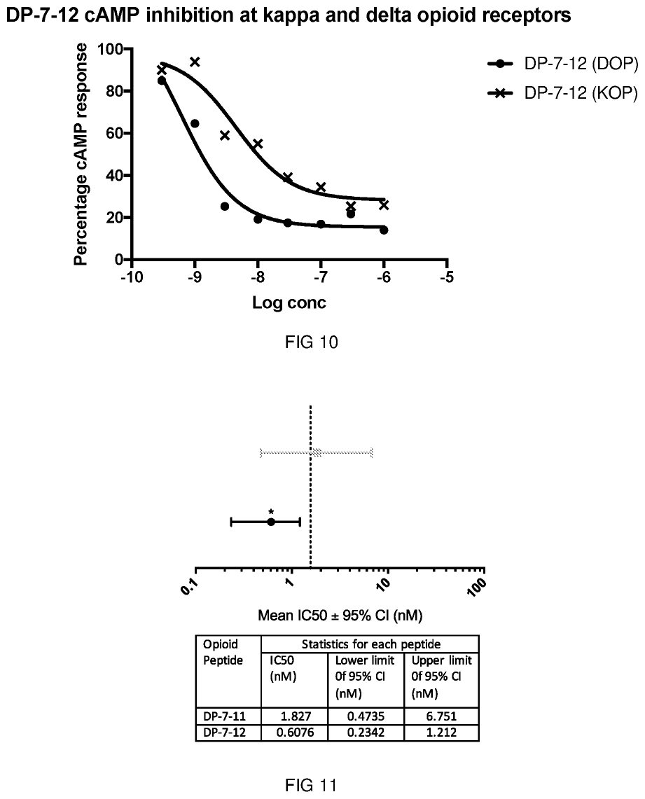

Concentration-response curves for DP-7-11 in HEK-DOP and HEK-KOP are shown in FIG. 9. Concentration-response curves for DP-7-12 in HEK-DOP and HEK-KOP are shown in FIG. 10. DP-7-11 achieved the higher maximum response (93.79%) and higher minimum response (23.13%) for HEK-DOP. In contrast, DP-7-12 achieved the lower maximum response (14.03%) for HEK-DOP. The IC50s (95% Cl) for the concentration-response curves are reported in FIG. 11. DP-7-12 had an IC50 of 0.6076 nM (0.2548 to 1.449 nM) and DP-7-11 had an IC50 of 1.827 nM (0.7474 to 4.468 nM) (FIG. 6). However, a F-test revealed that these IC50s were not statistically significant (FIG. 6, F=0.9098, p=0.4367).

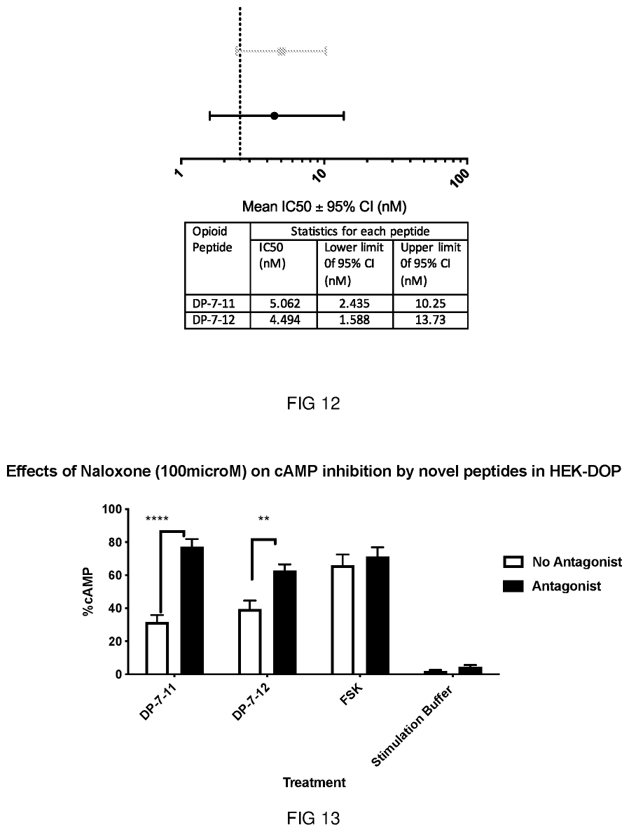

In regard to HEK-KOP, DP-7-11 and DP-7-12 display an inverse sigmoidal curve, with the % cAMP plateauing at a maximum at low concentrations of peptide and to a minimum at high concentration of peptide. Of cAMP responses, DP-7-12 attained the higher value (25.88%), whilst DP-7-11 attained the lower value (13.73%). The IC50s (95% Cl) for the concentration-response curves are reported in FIG. 12. DP-7-11 had a IC50 of 5.062 nM (2.435 to 10.25 nM) (FIG. 12). In addition, an F-test confirmed that these IC50s were statistically significant (FIG. 12, F=8.457, p<0.0001).

In comparing the IC50s between DOP and KOP (FIG. 11 and FIG. 12), an F-test confirmed no significant difference for DP-7-11 (F=2.021, p=0.1574). In contrast, an F-test reported a significant difference in DP-7-12 IC50 between DOP and KOP (FIGS. 6 and 7, F=10.44, p=0.0016).

Naloxone Reversal of Opioid Inhibition of Forskolin Induced cAMP

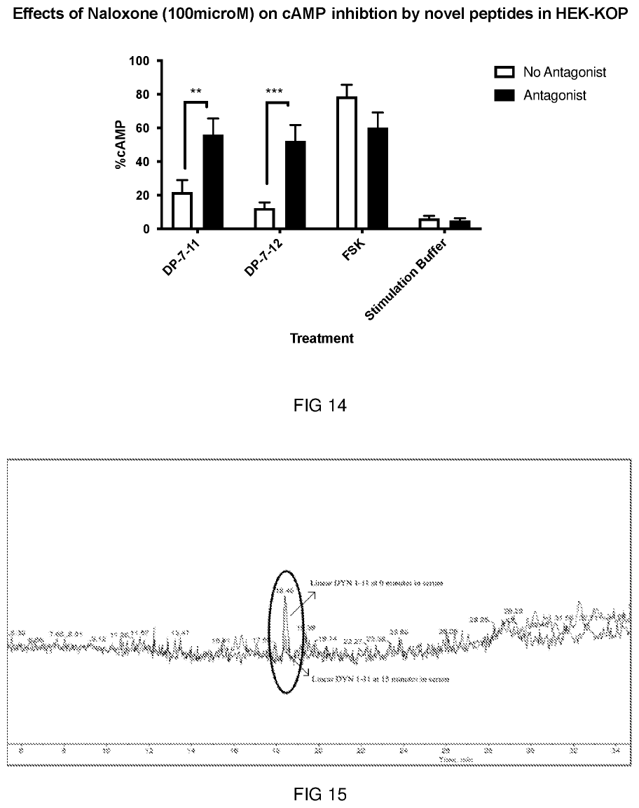

To confirm the specific receptor involvement of DP-7-11 and DP-7-12 with DOP, the cAMP assay was repeated to compare the cAMP response of HEK-DOP cells pre-treated with naloxone, with HEK-293 DOP cells in the absence of naloxone (FIG. 13). This was also done with HEK-KOP cells to confirm the activity of DP-7-11 and DP-7-12 on KOP (FIG. 14). In both DOP and KOP assays (FIG. 13 and FIG. 14), all cells were treated with DP-7-11 or DP-7-12 at the approximate IC80s (Table 9).

TABLE 9
IC80s of DP-7-11 and DP-7-12

| Cells | DP-7-11 | DP-7-12 |
|---|---|---|
| HEK-DOP | IC₈₀ = 24.09 nM | IC₈₀ = 2.723 nM |
| | (10.59 to 54.80 nM) | (1.892 to 3.917 nM) |
| HEK-KOP | IC₈₀ = 29.99 nM | IC₈₀ = 28.83 nM |
| | (17.72 to 50.78 nM) | (11.34 to 73.31 nM) |

FIGS. 13 and 14 illustrate the outcomes of antagonist addition for HEK-DOP and HEK-KOP respectively, including the effects of naloxone addition on cAMP response inhibition by DP-7-11 and DP-7-12, as well as both the positive and negative controls of the assay—Forskolin only and no Forskolin treatment respectively.

The inhibition of cAMP through DOP was reversed by naloxone for DP-7-11 and DP-7-12 (FIG. 13, p<0.05). DP-7-11 and DP-7-12 showed abilities to inhibit cAMP production to between 20 to 40% of maximal cAMP production in HEK-DOP cells with significant differences to the positive control (FIG. 13, p<0.05). DP-7-11 showed reversibility by naloxone, showing a mean cAMP response of 77.18%. DP-7-12 showed a cAMP response of 62.68% (FIG. 13). One-way ANOVA analysis found there to be no significant difference between both the Forskolin treatments with or without naloxone (FIG. 13, p>0.9999). Comparing each of the peptide-antagonist cAMP response with both the antagonist and without antagonist Forskolin treatments also found no significant difference (FIG. 8, p>0.9999).

The inhibition of cAMP through KOP was reversed by naloxone for DP-7-11 and DP-7-12 (FIG. 14, p<0.01). A significant difference was also found between the Forskolin only positive control—with and without naloxone- and the cAMP response for DP-7-11 and DP-7-12 (FIG. 9, p<0.05). Moreover, no significant difference was found between cAMP response for either DP-7-11 or DP-7-12 compared to both the no Forskolin antagonist and non-antagonist negative controls (FIG. 14, p<0.05). DP-7-11 and DP-7-12 showed mean cAMP responses of 56.14% and 52.40% respectively (FIG. 14).

### DISCUSSION

DP-7-11 and DP-7-12 are cyclic analogues of DP-7-00 aimed at reducing susceptibility to enzymatic metabolism and improve receptor selectivity. The cyclization of peptide molecules is a method of conferring enzymatic resistance. The rigidity of the ring structures, such as those formed in cyclization, are postulated to improve conformational variability which could translate to improved receptor selectivity and a reduction in off-target effects.

### Inhibitory Effects of DP-7-11 and DP-7-12

DP-7-11 and DP-7-12 showed opioid-like inhibitory activity at DOP and KOP (FIG. 9 and FIG. 10).

### Efficacy of Novel Opioid Peptides at DOP

It was found that DP-7-11 and DP-7-12 displayed no statistically different efficacies of cAMP inhibition at DOP (p>0.05). This supports the belief that DOPs are capable of adopting various conformations and thus accommodate a range of ligands.

### Efficacy of Novel Opioid Peptides at KOP

At KOP, DP-7-11 and DP-7-12 reported statistically significant differences in concentration-response curves (p<0.05) and IC50s (p<0.05). Like the results collected from DOP, DP-7-12 bettered DP-7-11 in potency in KOP. One reason for this difference in potency could be due to KOP itself. Known to have a clear difference in the position of its extracellular half of TM1 compared to DOP, the structure of KOP could be facilitating the specific location of the bulk found in DP-7-12. Previous research has shown that the removal of the N-terminal tyrosine residue by amino-peptidases abolishes the activity of Dynorphin at KOP. It therefore is possible that being closer to the tyrosine residue, the position of the bulky group in DP-7-11 has played a role in hindering the activity of the peptide compared to DP-7-12.

### DOP Vs KOP Efficacy

In agreement with previous findings of DP-7-00 equivalence at DOP and KOP, DP-7-11 reported no significant difference in IC50s between DOP and KOP subsets. For DP-7-11, these results support the hypothesis of equivalent potency in KOP and DOP.

DP-7-12 did not show statistically equivalent efficacy at DOP and KOP. The potency of DP-7-12 at DOP was ten times that of KOP.

### Comparison of Efficacy Between DP-7-11 and DP-7-12 with DP-7-00

DP-7-11 and DP7-12 vs. DP-7-00 at DOP

Although the IC50s of DP-7-11 and DP-7-12 at DOP showed no significant differences to DP-7-00 (p>0.05), it was found that the IC50 for DP-7-12 was an improvement on that of DP-7-00 (p<0.05). DP-7-12's terminally bulky structure may have allowed for reduced enzymatic metabolism of the essential peptide carboxy terminal while maintaining receptor access to the 1-Tyrosine residue, which is postulated to be vital for opioid activity. Nevertheless, DP-7-11 and DP-7-12 reported equivalent or better potency than DP-7-00, highlighting that the modifications present in these compounds succeeded in conserving efficacy.

DP-7-11 and DP7-12 vs. DP-7-00 at KOP

It is postulated that minimal modification to the DP-7-00 pharmacophore would maintain efficacy at the DP-7-00 level. That DP-7-11 and DP-7-12 did not report a change in potency (when compared with DP-7-00) suggests that cyclization at positions 2 and 5 had no significant effect on the potency for KOP.

### Reversibility of Novel Opioid Peptide Activity

Following the construction of the concentration-response curves for each cell line, naloxone was used to confirm the DOP and KOP receptor involvement in the modulation of Forskolin induced production of cAMP. Historically, naloxone has been characterised as a non-selective opioid receptor antagonist, with the capability to block the opioid modulated inhibition of intracellular cAMP production. In HEK-DOP, the addition of antagonist reversed the inhibitory activity of DP-7-11 and DP-7-12, to an equivalent cAMP response of the positive Forskolin only controls (FIG. 13). This is consistent with both naloxone's nature as a non-selective opioid antagonist and previous findings regarding the reversal of DP-7-00 activity in the DOP cAMP pathway. The positive reversibility of DP-7-11 and DP-7-12 in DOP highlights that the cyclic structure did not affect the mechanism of action in HEK-DOP cells. Reversal to the extent of the Forskolin only cAMP response via naloxone was found for the inhibitory actions of DP-7-11 and DP-7-12 in KOP (FIG. 14), further supporting previous discoveries confirming the reversibility of DP-7-00 mechanism of action in HEK-KOP.

The results of antagonist addition showed that DP-7-11 and DP-7-12 inhibited the cAMP response with significant difference to positive controls (p<0.05), to equivalent levels recorded by the no Forskolin control. These results indicate that the cyclization present in DP-7-11 and DP-7-12 may be protective.

TABLE 10
In vitro cAMP activity and stability of CP peptides

| | Activity (EC₅₀, nM) | | | Stability (t_{1/2}, mins) | |
|---|---|---|---|---|---|
| Peptide codes | KOP | MOP | DOP | Trypsin | Plasma |
| U50488 | 2 | >10000 | >10000 | n.a | n.a |
| Dyn1-17 | 1.5 | >10000 | >10000 | n.t | n.t |
| CP1 | 1269 | 4532 | >10000 | <0.01 | <0.01 |
| CP2 | 320 | 2327 | 1376 | <0.01 | <0.01 |
| CP3\hat{ } | >10000 | >10000 | >10000 | n.t | n.t |
| CP4\hat{ } | >10000 | >10000 | >10000 | n.t | n.t |
| CP5\hat{ } | >10000 | >10000 | >10000 | n.t | n.t |
| CP6 | 160 | 2.7 | tbd | <0.01 | 205.8 |
| CP7 | 36.5* | 937.5 | 757 | <0.01 | <0.01 |
| CP8 | 4.8* | 777.4 | 653.9 | <0.01 | <0.01 |
| CP9 | 1.8 | >10000 | >10000 | 49.8 | 85.5 |
| CP10 | 0.94 | 1602 | 127.2 | 2.3 | n.t |
| CP11 | 14.1 | tbd | tbd | 0.018 | <0.01 |
| CP12 | 7.5 | tbd | tbd | 0.019 | <0.01 |
| CP13 | 15.95 | >10000 | tbd | 1408 | 63.33 |
| CP14 | 4.4 | >10000 | tbd | 2325 | 70.83 |
| DP-11-06 | 0.75 | n.t | n.t | 360^{#&} | 30 |

n.t = not tested;
n.a = not applicable
tbd = to be determined
*partial agonist;
max activity <50% of U50488;
\hat{ }Not carried forward as found be of low potency in initial screening assay;
^{#}more than 90% remained after 360 minutes;
^{&}n = 1.

The results shown in Table 10 utilized rat plasma.
Note that DP-7-11 and CP5 represent the same compound, and DP-7-12 and CP6 are the same compound. In this regard, a different regression analysis method was adopted on the above cAMP experiments. Particularly, a new non-linear regression (four-parameter) was adopted to better account for hill slope, thereby improving the regression fit. This new method was applied to all existing data for accuracy and consistency, hence some numbers may vary.

### Further Plasma and Trypsin Stability Tests

Plasma was collected in house from adult mixed-gender Wistar rats, prepared using 2% EDTA as per standard practice. Peptides were added to rat plasma samples at 37° C. (in a water bath) with final concentrations of 100 uM (1:9 peptide in water:plasma) and a 50 μL sample was immediately taken and precipitated in 150 μL cold acetonitrile (9:1 ACN:water). This sample became the baseline, or t=0 min sample. Plasma with peptide was immediately returned to the water bath and subsequent 50 μL samples were taken at 5, 10, 15, 30, 60 and 120 min. At each time point, the 50 μL of plasma collected was immediately added to cold ACN. Each collected plasma sample in ACN was directly vortexed for 30 seconds and then centrifuged at room temperature (13K rpm, 5 min). 150 μL of the supernatant was taken and directly placed in glass HPLC vials for LCMS analysis.

The protocol for the trypsin stability assay was very similar to the plasma stability assay discussed above. The only difference was the use of a trypsin solution (bovine pancreatic trypsin 2.5 μg/mL in NH₄HCO₃ buffer, pH approx. 8-8.5, 37° C.) instead of rat plasma. Volumes, times and preparation protocols were exactly as mentioned above.

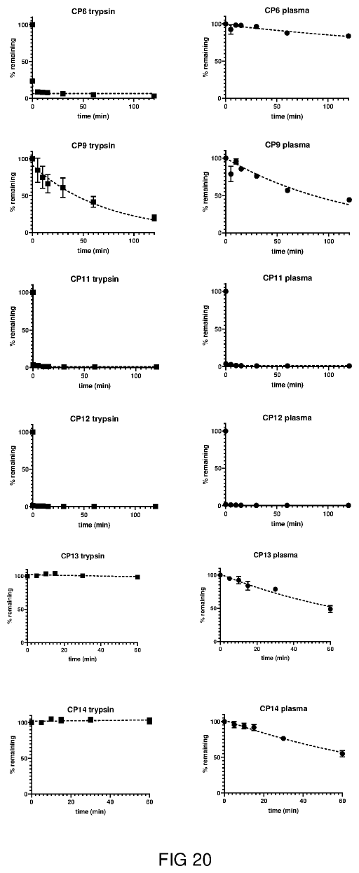

The in vitro plasma and trypsin stability data of the cyclic peptides is summarised in Table 10 (above), with representative figures shown in FIG. 20.

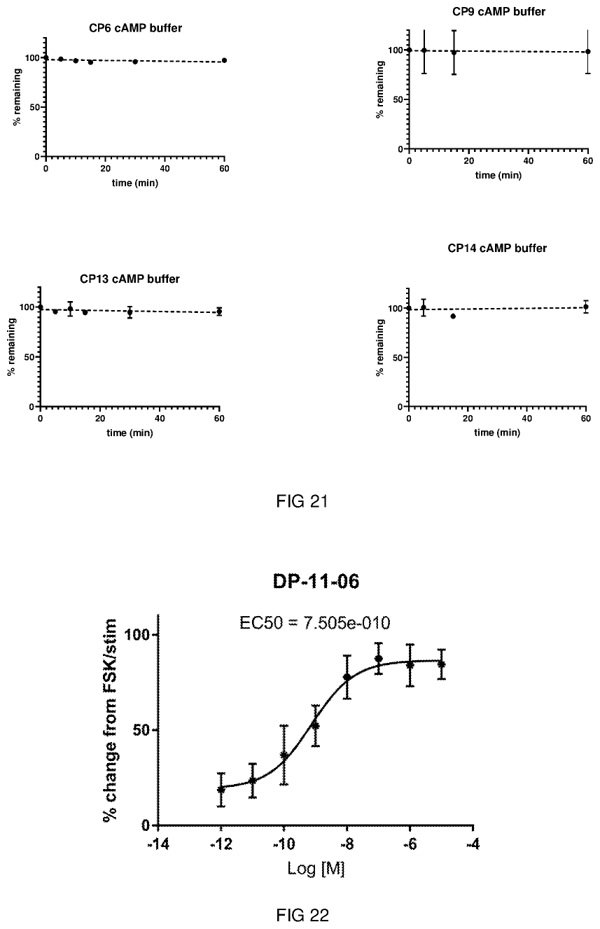

Select compounds were also screened for stability in cAMP buffer, to assess whether they degrade spontaneously in the cell assay environment, in the absence of cellular metabolic processes. All peptides screened (CP6, CP9, CP13 and CP14) showed no degradation over 60 minutes in the assay buffer (FIG. 21).

Candidates CP8, CP9, CP10, CP11, CP12, CP13 and CP14 show good potency in the cAMP assay, all being comparable to the potency of the reference compound, U50488, and the native/endogenous peptide, Dynorphin 1-17. This data suggests that this group of peptides possess characteristics that could make them clinically relevant analgesics (noted via cAMP EC50s). From a stability perspective, CP9 showed reasonable stability in both trypsin and plasma, where the cyclic structure was maintained. CP13 and CP14 showed exceptional stability in trypsin, with no sign of degradation over the 120 minute assay. These two peptides also had reasonable stability in plasma.

The data arising from this peptide series suggest that CP9, CP13 and CP14 are promising candidates for in vivo testing, based on potency in the cAMP assay and their intrinsic stability as cyclic peptides in trypsin and plasma. CP11 and CP12 also show good levels of potency.

It should be clear that compounds of the present invention are promising in the development of opioids with reduced side effects, as the targeting of the DOP/KOP receptors becomes a reality.

The above description of various embodiments of the present invention is provided for purposes of description to one of ordinary skill in the related art. It is not intended to be exhaustive or to limit the invention to a single disclosed embodiment. As mentioned above, numerous alternatives and variations to the present invention will be apparent to those skilled in the art of the above teaching. Accordingly, while some alternative embodiments have been discussed specifically, other embodiments will be apparent or relatively easily developed by those of ordinary skill in the art. Accordingly, this invention is intended to embrace all alternatives, modifications and variations of the present invention that have been discussed herein, and other embodiments that fall within the spirit and scope of the above described invention.

1. A compound of formula (I), or a salt or stereoisomer or solvate or prodrug thereof:
X₁-X₂-X₃-X₄-X₅-X₆-X₇-X₈-X₉-X₁₀-X₁₁ Formula (I)
wherein X₁, X₃, X₄, X₅, X₆ and X₇ are each independently an amino acid or derivative thereof; wherein X₂, X₈, X₉, X₁₀ and X₁₁, when present, are each independently an amino acid or derivative thereof; and
wherein a pair of any of X₁, X₂, X₃, X₄, X₅, X₆, X₇, X₈, X₉, X₁₀ and X₁₁ together form a linker 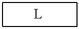
comprising 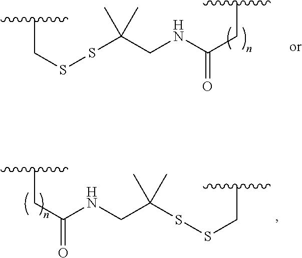
wherein n is 1 or 2.

2. The A-compound of claim 1, wherein
X₁, X₂, X₃, X₄, X₅, X₆ and X₇ are each independently an amino acid; wherein X₈, X₉, X₁₀ and X₁₁, when present, are each independently an amino acid; and
a pair of any of X₁, X₂, X₃, X₄, X₅, X₆, X₇, X₈, X₉, X₁₀ and X₁₁ together form a linker 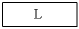
comprising 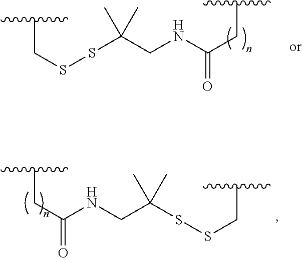
wherein n is 1 or 2.

3. The compound of claim 1, wherein:
If X₁ does not form part of the linker 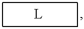
X₁ is tyrosine or a derivative thereof;
If X₄ does not form part of the linker 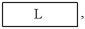
X₄ is phenylalanine or a derivative thereof;
If X₅ does not form part of the linker 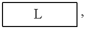
X₅ is selected from the group consisting of: leucine or a derivative thereof, isoleucine or a derivative thereof, and valine or a derivative thereof;
If X₆ does not form part of the linker 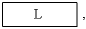
X₆ is arginine or a derivative thereof; and
If X₇ does not form part of the linker 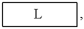
X₇ is arginine or a derivative thereof.

4. The compound of claim 1, wherein: X₁ is tyrosine or a derivative thereof; X₄ is phenylalanine or a derivative thereof; and X₆ is arginine or a derivative thereof.

5. The compound of claim 1, wherein X₈, X₉, X₁₀ and X₁₁ are not present.

6. The compound of claim 1, wherein 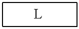
is formed between X₂ or X₃ and any remaining amino acid or derivative thereof.

7. The compound of claim 1, wherein 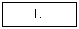
is formed between X₈ and X₁₀.

8. The compound of claim 1, wherein X₂ is not present.

9. The compound of claim 8, wherein X₃ is glycine or a derivative thereof.

10. A compound of formula (IX), or a salt or stereoisomer or solvate or prodrug thereof: 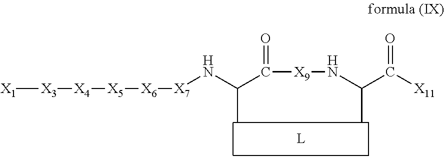
wherein X₁, X₃, X₄, X₅, X₆, X₇, X₉ and X₁₁ are each independently an amino acid or derivative thereof;
wherein 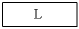
comprises 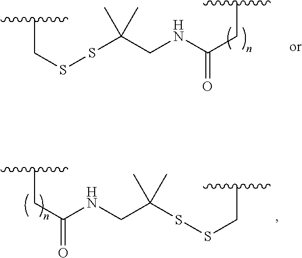
wherein n is 1 or 2.

11. The compound of claim 10, wherein:
X₁ is tyrosine or a derivative thereof;
X₄ is phenylalanine or a derivative thereof;
X₅ is selected from the group consisting of: leucine or a derivative thereof, isoleucine or a derivative thereof, and valine or a derivative thereof;
X₆ is arginine or a derivative thereof; and
X₇ is arginine or a derivative thereof.

12. The compound of claim 11, wherein X₃ is glycine or a derivative thereof.

13. The compound of claim 10, wherein:
X₁ is tyrosine;
X₃ is sarcosine;
X₄ is selected from the group consisting of: phenylalanine, p-nitrophenylalanine and p-chlorophenylalanine;
X₅ is leucine;
X₆ is arginine or N(cc)-methylarginine; and
X₇ is arginine or N(cc)-methylarginine.

14. The compound of claim 1, wherein the compound is selected from the group consisting of:
(DP-7-11) or (CP5)—c(Tyr-SSa-Gly-Phe-D(Asp)-Arg-Arg)
(DP-7-12) or (CP6)—c(Tyr-D(Asp)-Gly-Phe-SSa-Arg-Arg)
(DP-7-13) or (CP7)—c(Tyr-Gly-SSa-Phe-D(Asp)-Arg-Arg)
(DP-7-14) or (CP8)—c(Tyr-Gly-D(Asp)-Phe-SSa-Arg-Arg)
(DP-11-06)—c(Tyr-Gly-Gly-Phe-D(Asp)-Arg-SSa-Ile-Arg-Pro-Lys)
(CP1)—c(Tyr-SSa-Gly-Phe-L-Asp-Arg-Arg)
(CP2)—c(Tyr-Asp-Gly-Phe-SSa-Arg-Arg)
(CP3)—c(Tyr-Gly-SSa-Phe-Asp-Arg-Arg)
(CP4)—c(Tyr-Gly-Asp-Phe-SSa-Arg-Arg)
(CP9)—c(Tyr-Sar-(p-Cl-Phe)-Leu-Arg-D(Arg)-SSa-Asp)
(CP10)—c(Tyr-Sar-(p-Cl-Phe)-Leu-Arg-D(Arg)-SSa-Arg-Asp-Lys)
(CP11)—c(Tyr-Sar-(p-Cl-Phe)-Leu-Arg-NMA-SSa-Arg-Asp-Lys)
(CP12)—c(Tyr-Sar-(p-NO₂-Phe)-Leu-Arg-NMA-SSa-Arg-Asp-Lys)
(CP13)—c(Tyr-Sar(p-NO₂-Phe)-Leu-Arg-D(Arg)-SSa-Arg-D(Asp)-Lys and
(CP14)—c(Tyr-Sar-(p-NO₂-Phe)-Leu-Arg-NMA-D(Asp)-D(Arg)-SSa-D(Lys)),
wherein said compound is cyclized through the sidechains of SSa and Asp which together form the structure: 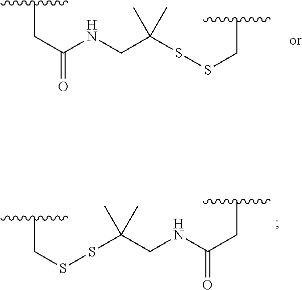 [SMILES: *CSSC(C)(C)CNC(=O)C* ; *CSSC(C)(C)CNC(=O)C*]
or a salt or stereoisomer or solvate thereof.

15. The compound of claim 1, wherein the compound is selected from the group consisting of: 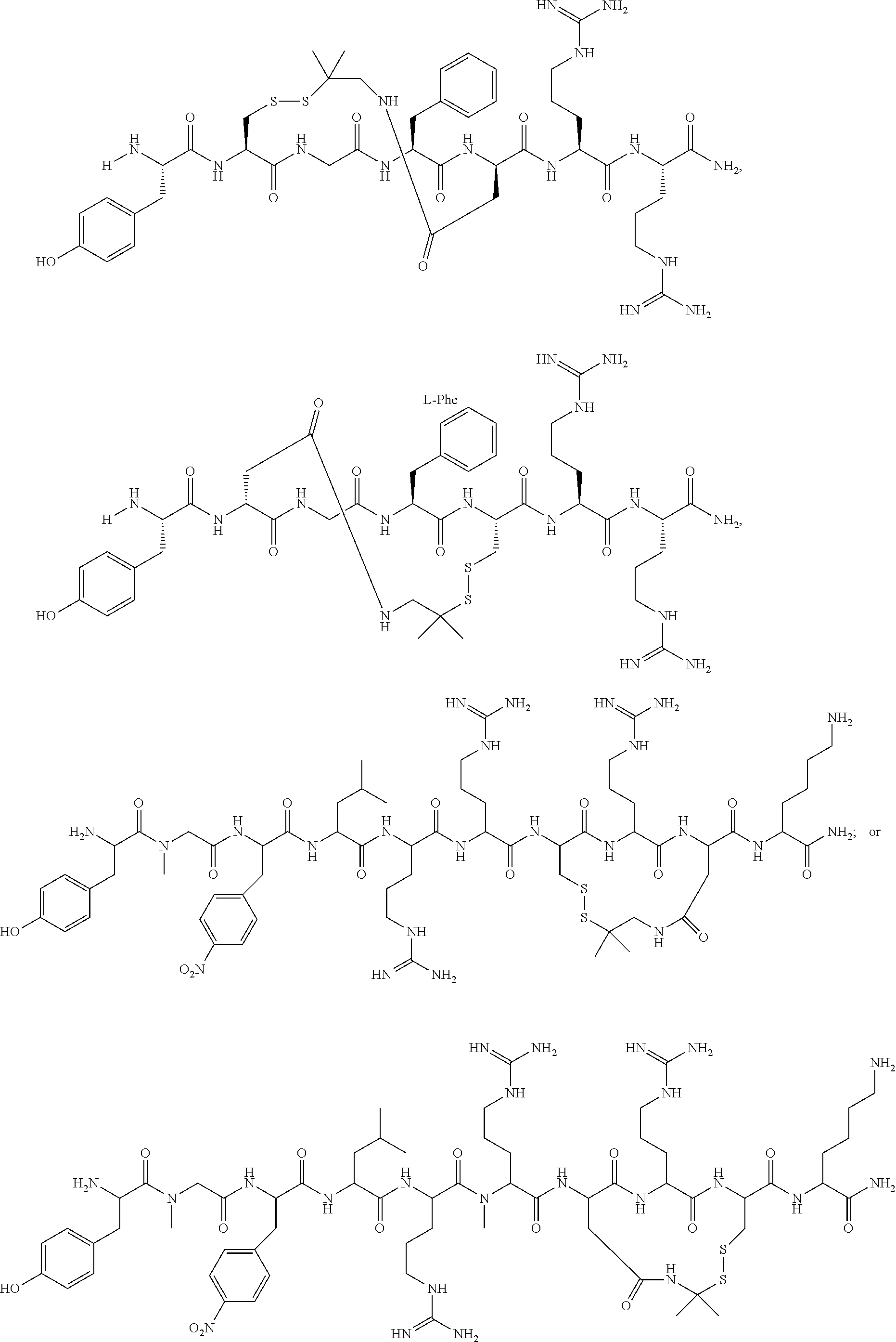
or a salt or stereoisomer or solvate or prodrug thereof.

16. A molecule comprising the compound of claim 1.

17. A pharmaceutical composition comprising the compound of claim 1, and a pharmaceutically acceptable carrier, diluent and/or excipient.

18. A method of treating or preventing pain in a subject including the step of administering a therapeutically effective amount of a compound of claim 1 to thereby treat or prevent pain.

19. The method of claim 18, wherein the subject is a mammalian subject.

20. (canceled)

21. The method of claim 18, wherein the pain is selected from the group consisting of nociceptive pain, somatic pain, visceral pain, neuropathic pain, pain syndrome, diabetic neuropathy, trigeminal neuralgia, postherpetic neuralgia, post-stroke pain, complex regional pain syndrome, reflex sympathetic dystrophy, causalgias, cancer pain, acute pain, chronic pain, inflammatory pain and psychogenic pain.
